(19) United States
(12) Patent Application Publication
(10) Pub. No.: US 2005/0142609 A1
(43) Pub. Date: Jun. 30, 2005

(54) Native immunoglobulin binding reagents and methods for making and using same

(75) Inventors: Brian Seed, Boston, MA (US); Gangzhou Li, San Diego, CA (US)

Correspondence Address: QUINE INTELLECTUAL PROPERTY LAW GROUP, P.C., P O BOX 458, ALAMEDA, CA, 94501, US

(73) Assignee: eBioscience, San Diego, CA (US)

(21) Appl. No.: 10/960,442

(22) Filed: Oct. 6, 2004

Related U.S. Application Data
(60) Provisional application No. 60/509,850, filed on Oct. 8, 2003.

(51) Int. Cl. G01N 33/53; C12N 5/06; C07K 16/42

(52) U.S. Cl. 435/7.1; 435/327; 530/387.2

(57) ABSTRACT

Isolated native immunoglobulin binding reagents including antibodies are provided, along with articles of manufacture, compositions and kits that include the native immunoglobulin binding reagents. Labeled reagents and substrates that comprise samples or the reagents are provided. Methods of screening for, making and using the reagents are also provided.

### CROSS REFERENCE TO RELATED APPLICATIONS

The subject application is a non-provisional of U.S. Ser. No. 60/509,850, entitled “NATIVE IMMUNOGLOBULIN BINDING REAGENTS AND METHODS FOR MAKING AND USING SAME” by Seed and Li, filed Oct. 8, 2003. The subject application claims priority to and benefit of U.S. Ser. No. 60/509,850, which is incorporated herein by reference in its entirety for all purposes.

### FIELD OF THE INVENTION

This invention relates generally to binding reagents that preferentially bind to native immunoglobulins over denatured immunoglobulins. This invention also relates generally to methods of producing these binding reagents and using them in a variety of research, diagnostic and therapeutic settings. These methods and binding reagents provide substantially improved and enhanced analysis of a wide variety of immunological interactions, including, but not limited to, those involving anti-antibody antibodies, such as analysis of immunoblotted proteins. This invention also relates to new antibodies and methods for making and using them, including, but not limited to, monoclonal and polyclonal antibodies, that preferentially bind to native immunoglobulin. This invention also relates to hybridomas producing such monoclonal antibodies as well as recombinant host cells expressing nucleic acid molecules encoding the antibodies. The present invention also relates to research, therapeutic and diagnostic methods and compositions employing these antibodies and binding reagents, as well as kits containing them.

### BACKGROUND OF THE INVENTION

One of the first demonstrations of monoclonal antibody production was done in 1975 by Kohler and Milstein [256 NATURE 495-497 (1975)]. Much effort has since been directed to the production of various hybrid cells (called “hybridomas”) and to the use of the antibody made by these hybridomas for various scientific investigations. See, for example, U.S. Pat. No. 4,515,893 entitled “Hybrid Cell Line for Producing Complement-Fixing Monoclonal Antibody to Human T Cells” illustrating some of the rewards, complications and variations of attempting to produce monoclonal antibody from hybridomas. Production of monoclonal antibodies is influenced by the types of antigens used as well as the selection methods employed for isolation of the desired hybridoma.

The immune system tends to mount a response toward the more abundant, immunodominant epitopes in a protein mixture; therefore, these traditional monoclonal antibody production techniques frequently result in the generation of monoclonal antibodies against immunodominant epitopes. When trying to produce antibodies specific for proteins that are rare or poorly immunogenic, difficulty often arises. In addition, isolation of antibodies specific for a protein with significant sequence similarity to other proteins can also be challenging. Subtractive immunization is an established technique utilized for the generation of antibodies specific for antigens that less abundant, poorly immunogenic and/or similar in sequence or structure to other proteins [Zijlstra et al., “Targeting the Proteome/Epitome, Implementation of Subtractive Immunization”, 303(3) BIOCHEM. BIOPHYS. RES. COMMUN., 733-744 (2003); Lian-June Yang and Wen-Liang Wang, “Preparation of Monoclonal Antibody Against Apoptosis-Associated Antigens of Hepatoma Cells by Subtractive Immunization”, 8(5) WORLD J. GASTROENTEROL, 808-814 (2002)].

Purification and identification of a target protein from a crude protein mixture, such as a cellular lysate, is generally achieved by immunoprecipitation followed by immunoblotting. The immunoprecipitation technique utilizes specific antibodies bound to an insoluble substrate, such as a resin, to capture and precipitate the protein of interest. Unbound proteins are removed by centrifugation and the protein of interest is recovered from the solid support by an elution buffer which also denatures and releases the antibody bound to the resin.

The classical immunoprecipitation procedure commonly used in cell biology research results in the separation of the immunoprecipitated protein band by SDS-PAGE analysis. However, the proteins assessed by SDS-PAGE also contain heavy and light chains from the denatured antibody that was released from the resin. This complicates the analysis of the purified protein, especially when the SDS-PAGE separation is followed by immunoblotting (e.g., western blotting) where the labeled secondary antibodies used to reveal the blotting antibody will also react with the denatured heavy and light chains of the immunoprecipitating antibodies. Data interpretation is further hampered when the molecular weight of the protein of interest is close to the antibody heavy or light chains and the protein is therefore masked by their presence.

Proteomics research relies largely on the classical immunoprecipitation/immunoblotting techniques for many studies, such as protein expression patterns, protein-protein interactions, post-translational modifications and protein function. Presently available techniques and products attempting to eliminate or minimize the impact of the presence of the denatured heavy and light chains on immunoblots have been unsatisfactory, have found limited use, and have not offered a good solution to these problems. “Seize X” from Pierce Chemical of Rockford, Ill. uses a cross linker, DSS, to cross link primary antibody to Protein A or G on a bead, properly orienting the antibody so that the antigen-binding site faces away from the protein A or G support. This immobilization technique allows researchers the possibility of reusing the primary antibody and reducing heavy and light chain contamination in their final sample. The Seize X kit's disadvantages including factors such as: (i) time—an additional hour is required to use this for enhancement of immunoprecipitations, and (ii) complexity—additional steps are required to be added to the procedures.

Prior to the subject invention, no reagents were available that preferentially recognize native immunoglobulin for this purpose. The subject invention provides such reagents and other features that will be apparent upon a complete review of the following.

### SUMMARY OF THE INVENTION

The present invention provides, in one embodiment, novel binding reagents and methods utilizing them, to preferentially detect native immunoglobulin. These binding reagents and methods of making and using them provide substantially enhanced results in a wide variety of research, diagnostic and therapeutic methods, including but not limited to, any method where detection of native antibody molecules is involved, such as in procedures for detection of immunoblotted protein. The binding reagents of the present invention and methods utilizing them are also useful in a wide variety of research techniques and diagnostic procedures, whenever anti-antibody antibodies are being used.

These binding reagents are referred to herein as native immunoglobulin-specific binding reagents (NIgSBR). NIgSBR can be any type of molecule or compound, including, but not limited to, small organic and inorganic molecules, proteins, peptides, antibodies, nucleic acids, polysaccharides, or any other molecule that specifically binds to native immunoglobulin. For example, the NIgSBR can be a polyclonal antibody, a monoclonal antibody, an antibody portion, an antibody fragment, an antibody variant, an engineered protein, a polymer scaffold, an engineered compound, a polypeptide, a polymer made in a mammalian system, a polymer made in a non-mammalian system, a polymer made in an E. coli by phage display, or the like.

These NIgSBR can be produced, e.g., by screening one or more compounds to identify a compound that specifically binds to native immunoglobulin. For example, the NIgSBR can be identified from a library, such as an antibody library, a protein scaffold library, a peptide display library, a directed evolution library, a protein array-based library, or the like. A composition comprising the NIgSBR can include the NIgSBR and other materials, such as diluents, adjuvants, carriers, or the like, depending on the use for the composition. The NIgSBR can be labeled, e.g., with a chemiluminescent agent, a radioisotope, an enzyme, a fluorescent agent, a chromogenic agent, or the like. Similarly, articles of manufacture (e.g., kits for research or diagnostic use) that include the NIgSBR can additionally include packaging materials and a container comprising the composition. immobilized on a substrate.

The present invention includes methods for identifying and isolating NIgSBRs, providing NIgSBRs that have a binding specificity that allows the detection and/or quantitation of native immunoglobulin. Either liquid phase or solid phase detection methods can be used with the NIgSBRs of the invention. For example, in various preferred detection formats, a NIgSBR or a sample that is being detected by the NIgSBR can be affixed to a solid substrate, e.g., a bead, a plate, a sheet, a strip, a well, a tube or the like. Alternately, a liquid phase detection format, e.g., followed by electrophoresis of bound components can be used.

As noted, the present invention provides NIgSBR, including, in preferred aspects, novel antibodies and methods for producing them, for use in procedures to preferentially detect native immunoglobulin. For example, the invention provides a method for producing at least one native immunoglobulin-specific binding reagent, such as an anti-native immunoglobulin-specific antibody (in this case the relevant NIgSBR is an “anti-NigSAb”), by screening antibodies raised against an immunoglobulin antigen to identify antibodies that bind specifically to a native immunoglobulin. The antibody can take any of the forms noted herein and can be used in any of the detection formats noted for NIgSBR in general. These antibodies provide substantially enhanced results in a wide variety of research, diagnostic and therapeutic methods, including but not limited to, any method where detection of native antibody molecules is involved, such as in procedures for detection of immunoblotted proteins. The methods and antibodies of the present invention are also useful for a wide variety of research techniques and also diagnostic procedures, e.g., whenever anti-antibody antibodies are used. The unique ability of the methods and antibodies of the present invention to specifically bind to native immunoglobulin provides substantially improved results when used for immunological methodologies, including, but not limited to, immunoprecipitation and Western blotting techniques.

Accordingly, homogeneous antibody preparations, e.g., that include isolated anti-NIgSAb having an ability to distinguish between immunoglobulin in their native versus denatured state are provided. An antibody of the invention can be, e.g., a monoclonal antibody, a polyclonal antibody, an antibody portion, an antibody fragment, an antibody variant, an anti-NIgSAb, an anti-NIgSAb portion, an anti-NIgSAb fragment, or an anti-NIgSAb variant. The antibody can be raised in a mammal, such as a human, primate, rodent, mouse, rat, hamster, rabbit, horse, donkey, sheep, or goat, and/or can be a chimeric, humanized and/or can be a CDR-grafted anti-NigSAb.

For example, the antibody can be raised in the mammal by subtractive immunization that includes administration of a denatured immunoglobulin followed by immunization with a native immunoglobulin. The resulting polyclonal antibody can be subtracted with denatured immunoglobulin, e.g., attached to an insoluble substrate (e.g., a bead, plate, well, tube, membrane, or sheet.

Compositions that include the anti-NigSAb and a suitable diluent, adjuvant, or carrier are also provided. Cleavage products and other specified portions and variants thereof, as well as anti-NIgSAb compositions, encoding or complementary nucleic acids, vectors, host cells, cell lines for producing anti-NigSAb, compositions, formulations, devices, articles of manufacture, such as kits, transgenic animals, transgenic cells, transgenic plants, and methods of making and using thereof, as described and enabled herein, and in combination with what is known in the art.

For example, the invention provides an article of manufacture, e.g., for research or diagnostic use, comprising packaging material and a container comprising at least one NIgSBR, e.g., an isolated anti-NIgSAb. The NIgSBR, such as an anti-NigSAb, is typically detectably labeled to facilitate use of the article. Any available label can be used, e.g., a chemiluminescent agent, a radioisotope, an enzyme, a fluorescent agent, a chromogenic agent, or the like. In one example, the label comprises an enzyme that produces a detectable product. For example, the enzyme can be a HRP enzyme. An article of manufacture that is formatted as a kit can additionally include, e.g., instructions for using the anti-NigSAb or other NIgSBR, control reagents, diluents, or the like.

At least one antibody of the invention binds at least one specified epitope specific to at least one native immunoglobulin protein, subunit, conformation, fragment, portion or any combination thereof, including, but not limited to, any epitope found on native heavy or light chain from any class or isotype of immunoglobulin molecule. At least one epitope can comprise at least one antibody binding region that comprises at least one portion of said protein, which said epitope may be linear or conformational, and may be comprised of 1 to 5 or 5 or more amino acids of at least one portion thereof, such as but not limited to, at least one domain, linear or conformational, found in the native form of said immunoglobulin protein, or any portion thereof. A linear epitope comprising a contiguous sequence of amino acids, and a conformation epitope comprises amino acids that may not be contiguous but are formed from primary, secondary, tertiary or quaternary structure of the antigen. At least one antibody of the present invention binds preferably to non-denatured immunoglobulin.

At least one anti-NIgSAb in another embodiment of the present invention can optionally comprise at least one specified portion of at least one complementarity determining region (CDR) (e.g., CDR1, CDR2 or CDR3 of the heavy or light chain variable region) and/or at least one constant or variable framework region or any portion thereof. The anti-NIgSAb amino acid sequence can further optionally comprise at least one specified substitution, insertion or deletion as described herein or as known in the art.

The present invention provides at least one isolated monoclonal anti-NIgSAb as described herein, wherein the antibody has at least one activity such as, but not limited to, the ability to preferentially bind to native immunoglobulin molecules. An anti-NIgSAb, whether monoclonal or polyclonal, can thus be screened for a corresponding activity according to known methods.

In another embodiment of the present invention, methods for preparing: (i) hybridomas producing such antibodies; (ii) polyclonal antibodies; and (iii) other binding reagents that bind preferentially to native immunoglobulin, are provided.

In a related embodiment, the present invention also provides at least one method for expressing at least one anti-NIgSAb, in a hybridoma or a recombinant host cell, comprising culturing a hybridoma or a recombinant host cell as described herein under conditions wherein anti-NIgSAb is expressed in detectable and/or recoverable amounts. The recombinant host cell expressing the anti-NIgSAb comprises a nucleic acid molecule encoding the anti-NIgSAb.

In another embodiment, the present invention also provides at least one composition comprising: (a) one or more isolated anti-NIgSAb encoding nucleic acid, recombinant host cell, anti-NIgSAb, NIgSBR, and/or hybridoma as described herein; and (b) a suitable carrier or diluent. The carrier or diluent can optionally be reagent grade or pharmaceutically acceptable, according to known carriers or diluents, and may also be dried or lyophilized. The composition can optionally further comprise at least one additional compound, protein or composition. In one embodiment of the present invention also includes a kit comprising one or more of these compositions.

Utilizing the NIgSBR's and methods of the present invention, contaminating denatured antibody that may be, for example, carried over from standard immunoprecipitation procedures, does not interfere with the final immunoblotting steps. The NIgSBR and anti-NIgSAb of the present invention substantially enhance detection of a protein of interest on an immunoblot and substantially improves data interpretation and presentation. The NIgSBR, anti-NIgSAb, and methods of the present invention for making and using them, are suitable in a wide variety of research, therapeutic and diagnostic procedures, including, but not limited to, procedures in which there may be native and denatured immunoglobulin molecules present.

The methods of the present invention do not add extra steps to the commonly utilized immunoblotting protocols and do not require modifications to the procedures commonly used in cell biology and proteomics research laboratories. For example, while the denatured heavy and light chains of the primary antibodies carried over from the immunoprecipitation steps may be present on the immunoblot, they are not detected, or are not detected as much, by the NIgSBR or anti-NIgSAb of the present invention. The NIgSBR, anti-NIgSAb, and methods of the present invention substantially simplify and enhance the analysis of the immunoblotted proteins by eliminating the detection of the denatured antibody.

The present invention also provides, in one related aspect, isolated nucleic acid molecules comprising, complementary, or hybridizing to, a polynucleotide encoding specific NIgSBR such as anti-NIgSAb, comprising at least one specified sequence, domain, portion or variant thereof. The present invention further provides recombinant vectors comprising said anti-NIgSAb nucleic acid molecules, host cells containing such nucleic acids and/or recombinant vectors, as well as methods of making and/or using such antibody nucleic acids, vectors and/or host cells.

These and a wide variety of additional embodiments of the present invention are readily apparent to those of ordinary skill in the art as disclosed herein.

### BRIEF DESCRIPTION OF THE FIGURES

FIG. 1: Shows the preferential binding of antibodies of the present invention to native immunoglobulin in Western blot format.

FIG. 2, Panels A and B: Shows the result of the methods of the present invention producing and selecting for antibodies that preferentially bind to native immunoglobulin in Western blot format.

FIG. 3: Shows the result of Western blotting using conventional antibodies that do not preferentially bind to native immunoglobulin (Lane 1) compared with an anti-NIgSAb of the present invention (Lane 2). Lane 3 shows Lane 2 re-blotted with the conventional antibody used in Lane 1.

### DETAILED DESCRIPTION OF THE INVENTION

The present invention provides isolated NIgSBR such as anti-NIgSAb, as well as methods for making them, compositions and kits comprising them, and nucleic acid molecules encoding the anti-NIgSAb. The present invention further includes, but is not limited to, methods of using the NIgSBR and anti-NIgSAb of the present invention, including research, therapeutic and diagnostic methods and devices. The NIgSBR and anti-NIgSAb of the present invention bind specifically to native immunoglobulin.

By “binds specifically” is meant high avidity and/or high affinity binding of a binding reagent such as an antibody to a specific polypeptide e.g., epitope, of a native immunoglobulin protein. Antibody binding to an epitope on this specific polypeptide is preferably stronger than binding of the same antibody to any other epitope, particularly those which may be present in molecules in association with, or in the same sample as the specific polypeptide of interest, e.g., the antibody binds more strongly to a native immunoglobulin than denatured forms or fragments of immunoglobulin, so that, by adjusting binding conditions, the antibody binds more preferentially to native immunoglobulin and less preferentially to denatured forms or fragments of the immunoglobulin. Antibodies which bind specifically to a native immunoglobulin polypeptide of interest can be capable of binding other polypeptides, such as denatured immunoglobulin, at a weak, yet detectable, level (e.g., 50% or less, 40% or less, 30% or less, 20% or less, 15% or less, 10% or less, 5% or less, 1% or less, or 0.1% or less, of the binding shown for the native immunoglobulin polypeptide of interest. Such a binding differential, or, alternatively, background binding, is readily discernible from specific antibody binding to the native immunoglobulin polypeptide of interest, e.g. by use of appropriate controls. However, it is readily apparent to those of ordinary skill in the art that any detectable difference between the binding to native immunoglobulin and denatured immunoglobulin demonstrates the preferential binding to the native immunoglobulin of interest. In general, anti-NIgSAb of the present invention which preferentially bind to native immunoglobulin with a binding affinity of about 10⁶ mole/liter or more, or about 10⁷, or about 10⁸ mole/liter or more, bind specifically to native immunoglobulin.

An “isolated” biological component such as an anti-NigSAb is one that is partially or completely purified away from the biological components that it is produced with or by. For example, the anti-NigSAb can be partially purified away from cellular materials that are used in the production of the anti-NigSAb. The term isolated does not require complete purification to homogeneity; rather, the relevant component is typically purified to an extent sufficient to be useful in a relevant assay. The anti-NigSAb of the invention can also be considered “recombinant” which indicates that the anti-NigSAb or coding material thereof (e.g., a recombinant nucleic acid, gene, polynucleotide, etc.) has been produced or altered by human intervention. Generally, the arrangement of parts of a recombinant molecule is not a native configuration, or the primary sequence of the recombinant polynucleotide or polypeptide has in some way been manipulated. The alteration to yield the recombinant material can be performed on the material within or removed from its natural environment or state. For example, a naturally occurring nucleic acid becomes a recombinant nucleic acid if it is altered, or if it is transcribed from DNA which has been altered, by means of human intervention performed within the cell from which it originates. A gene sequence open reading frame is recombinant if that nucleotide sequence has been removed from its natural context and cloned into any type of artificial nucleic acid vector. Protocols and reagents to produce recombinant molecules, especially recombinant nucleic acids, are common and routine in the art. The term “recombinant” can also refer to an organism that harbors recombinant material, e.g., a cell, plant or animal that comprises a recombinant nucleic acid is considered a recombinant cell, plant or animal. In some embodiments, a recombinant organism is a transgenic organism.

The NIgSBR and anti-NIgSAb of the present invention can optionally be detectably labeled to provide a detectably labeled NIgSBR or detectably labeled anti-NIgSAb. By “detectably labeled”, “detectably labeled NIgSBR” or “detectably labeled anti-NIgSAb” is meant any substance (an antibody or antibody fragment or any other compound which retains binding specificity for native immunoglobulin), having an attached detectable label. The detectable label is normally attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling compounds such as antibodies, and methods for using labeled antibodies are well known in the art (see, for example, Harlow and Lane, eds. (Antibodies: A Laboratory Manual, Cold Spring Harbor Laboratory Press, Cold Spring Harbor, N.Y., 1988)).

The NIgSBR of the present invention can be any type of substance, including proteins, antibodies, peptides, nucleic acids, carbohydrates, polysaccharides, and portions or fragments thereof, or any other organic or inorganic molecule that binds specifically to native immunoglobulin.

An antibody according to the present invention includes any protein or peptide containing molecule that comprises at least a portion of an immunoglobulin molecule, such as, but not limited to, at least one complementarity determining region (CDR) of a heavy or light chain or a ligand binding portion thereof, a heavy chain or light chain variable region, a heavy chain or light chain constant region, a framework region, or any portion thereof, that can be incorporated into an antibody of the present invention. An antibody of the present invention can include or be derived from any animal, including mammals, such as, but not limited to, a human, a mouse, a rabbit, a rat, a rodent, a goat, a primate, or any combination thereof, and the like.

Binding Reagent or Antibody/Antigen Binding Forces

The forces which hold an antigen and antibody (or any binding reagent) together are in essence no different from non-specific interactions which occur between any two unrelated proteins e.g., other macromolecules such as human serum albumin and human transferrin. These intermolecular forces are typically classified into four general areas which include (1) electrostatic; (2) hydrogen bonding; (3) hydrophobic; and (4) Van der Waals. Electrostatic forces are due to the attraction between oppositely charged ionic groups on two protein side-chains. The force of attraction (F) is inversely proportional to the square of the distance (d) between the charges. Hydrogen bonding forces are provided by the formation of reversible hydrogen bridges between hydrophilic groups such as —OH, —NH₂ and —COOH. These forces are largely dependent upon close positioning of two molecules carrying these groups. Hydrophobic forces operate in the same way that oil droplets in water merge to form a single large drop. Accordingly, non-polar, hydrophobic groups such as the side-chains on valine, leucine and phenylalanine tend to associate in an aqueous environment. Lastly, Van der Waals are forces created between molecules which depend on interaction between the external electron clouds.

Further information regarding each of the different types of forces can be obtained from “Essential Immunology” edited by I. M. Roitti (6th Edition) Blackwell Scientific Publications, 1988. With respect to the present invention, NIgSBR or anti-NIgSAb exhibit some or all of these forces. It is by obtaining an accumulation of these forces in larger amounts that one may obtain a NIgSBR or an anti-NIgSAb which has a high degree of affinity or binding strength to the native immunoglobulin protein. One of ordinary skill in the art may readily screen a wide variety of compounds or antibodies for the specific binding to native immunoglobulin using the methods herein in conjunction with that which is generally available in the art.

Measuring Binding Reagent or Antibody/Antigen Binding Strength

The binding affinity between a NIgSBR or an anti-NIgSAb and native immunoglobulin can be measured and is an accumulation of a measurement of all of the forces described above. Standard procedures for carrying out such measurements are well known to those of ordinary skill in the art and can be directly applied to measure the affinity of any compound or antibodies of the present invention for native immunoglobulin.

One standard method for measuring antibody/antigen binding affinity is through the use of a dialysis tubing which is a container comprised of a material which is permeable to the antigen but impermeable to the antibody. This method is also useful for other binding reagents that are not antibodies, such as a non-antibody NIgSBR of the present invention. Antigens which are bound completely or partially to antibodies are placed within the dialysis tubing in a solvent such as water. The tubing is then placed within a larger container which does not contain antibodies or antigen but contains only the solvent e.g., the water. Since only the antigen can diffuse through the dialysis membrane of the tubing, the concentration of the antigen within the dialysis tubing and the concentration of the antigen within the outer larger container will begin to equilibrate. After placing the dialysis tubing into the larger container and allowing for time to pass towards reaching an equilibrium, it is possible to measure the concentration of the antigen within the dialysis tubing and within the surrounding container and then determine the differences in concentration. This makes it possible to calculate the amount of antigen which remains bound to antibody in the dialysis tubing and the amount which disassociates from the antibody and diffuses into the surrounding container. By constantly renewing the solvent (e.g., the water) within the surrounding container so as to remove any antigen which is diffused thereinto it is possible to totally disassociate the antibody from antigen within the dialysis tubing. If the surrounding solvent is not renewed the system will reach an equilibrium and it is possible to calculate the equilibrium constant (K) of the reaction i.e., the association and disassociation between the antibody and antigen. The equilibrium constant (K) is calculated as is an amount equal to the concentration of antibody bound to antigen within the dialysis tubing divided by the concentration of free antibody combining sites times the concentration of free antigen. The equilibrium constant or “K” value is generally measured in terms of liters per mole. The K value is a measure of the difference in free energy between the antigen and antibody in the free state as compared with the complexed form of the antigen and antibody.

Binding Reagent or Antibody Avidity

As indicated above, the term “affinity” describes the binding of a binding reagent such as an antibody to a single antigen determinate. However, frequently, one is concerned with the interaction of an antibody with a multivalent antigen. The term “avidity” is used to express this binding. Factors that contribute to avidity are complex and include the heterogeneity of the antibodies in a polyclonal sample, which are directed against more than one determinate on an antigen and against heterogeneity of the determinants themselves. The multivalence of most antigens leads to an effect in which the binding of two antigen molecules by an antibody is greater, and optionally many fold greater, than the arithmetic sum of the individual antibody interactions. Thus, it can be understood that the measured avidity between an antiserum and a multivalent antigen will be somewhat greater than the affinity between an antibody and a single antigen determinate.

As used herein, the term “epitope” refers to that portion of any molecule capable of being recognized by and bound to a binding reagent or an antibody at one or more of the antigen binding regions. Epitopes can be any molecule or grouping thereof, including, but not limited to, amino acids and side chains of sugars, and can have a specific three-dimensional structure or conformation. An epitope can comprise any portion of a protein molecule that includes primary, secondary, tertiary or quaternary structure, as those terms are generally used in the art.

As used herein, an “anti-NIgSAb”, “anti-NIgSAb portion”, or “anti-NIgSAb fragment” and/or “anti-NIgSAb variant” and the like include any protein or peptide containing molecule that comprises at least a portion of an immunoglobulin molecule, such as, but not limited to, at least one complementarity determining region (CDR) of a heavy or light chain or a ligand binding portion thereof, a heavy chain or light chain variable region, a heavy chain or light chain constant region, a framework region, or any portion thereof. As a non-limiting example, a suitable anti-NIgSAb, specified portion or variant of the present invention can bind at least one native immunoglobulin molecule, or specified portions, variants or domains thereof. The term anti-NIgSAb or “antibody” is further intended to encompass antibodies, antibody digestion fragments, specified antibody portions and variants thereof, including antibody mimetics, or portions of antibodies that mimic the structure and/or function of an antibody or specified fragment or portion thereof, including single chain antibodies and fragments thereof. Functional fragments include antigen-binding fragments that bind to a non-denatured immunoglobulin molecule. For example, antibody fragments capable of binding to a native immunoglobulin molecule or portions thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and re-aggregation), Fv or scFv (e.g., by molecular biology techniques) fragments, are encompassed by the present invention. See also, Paul (ed.) FUNDAMENTAL IMMUNOLOGY, FOURTH EDITION, Lippincott-Raven, NY, N.Y. (1999), incorporated herein in its entirety.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding an F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

Bispecific, heterospecific, heteroconjugate or similar antibodies can also be used that are monoclonal antibodies that have binding specificities for at least two different antigens. In the present case, one of the binding specificities is for at least one native immunoglobulin molecule or portion thereof, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, 305 NATURE, 537 (1983)). Because of the random assortment of immunoglobulin heavy and light chains, these hybridomas (quadromas) produce a potential mixture of ten different antibody molecules, of which only one has the correct bispecific structure. Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759; 5,601,819; 5,582,996; 5,496,549; 4,676,980; WO 91/00360, WO 92/00373, EP 03089, Traunecker et al., 10 EMBO J., 3655 (1991), Suresh et al., 121 METHODS IN ENZYMOLOGY, 210 (1986), each entirely incorporated herein by reference.

Antibodies of the Present Invention

At least one anti-NIgSAb of the present invention can optionally be produced by a cell line, a mixed cell line, an immortalized cell or clonal population of immortalized cells, as well known in the art. See, e.g., Ausubel et al. (Ed.), Current Protocols in Molecular Biology, (John Wiley & Sons, Inc., New York, N.Y. (1987-2001)); Sambrook et al., Molecular Cloning: A Laboratory Manual, 2^{nd} Edition, (Cold Spring Harbor, N.Y. (1989)) and Sambrook et al., Molecular Cloning—A Laboratory Manual (3rd Ed.), Vol. 1-3, Cold Spring Harbor Laboratory, Cold Spring Harbor, N.Y., 2000 (collectively, “Sambrook”); Harlow and Lane, Antibodies, A Laboratory Manual, (Cold Spring Harbor, N.Y. (1989)); Colligan, et al. (Eds.), Current Protocols in Immunology, (John Wiley & Sons, Inc., N.Y. (1994-2001)); Colligan et al., Current Protocols in Protein Science, (John Wiley & Sons, NY, N.Y., (1997-2001)), each entirely incorporated herein by reference.

Anti-NIgSAb's or fragments, portions and variants thereof can be raised against an appropriate immunogenic antigen, such as isolated immunoglobulin protein or a portion thereof (including synthetic molecules, such as synthetic peptides) and can be polyclonal or monoclonal antibodies. Other specific or general mammalian antibodies can be similarly raised. Preparation of immunogenic antigens, as well as polyclonal and monoclonal antibody production can be performed using any suitable technique known to those of ordinary skill in the art.

In one approach for preparing monoclonal antibodies, a hybridoma is produced by fusing a suitable immortal cell line (e.g., a myeloma cell line) such as, but not limited to, Sp2/0, Sp2/0-AG14, P3/NS1/Ag4-1, P3X63Ag8.653, MCP-11, S-194, or the like, or heteromyelomas, fusion products thereof, or any cell or fusion cell derived therefrom, or any other suitable cell line as known in the art. See, e.g., www.atcc.org, www.lifetech.com, or the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, Chapter 2, entirely incorporated herein by reference.

Antibody producing cells can also be obtained from the peripheral blood or, preferably, the spleen or lymph nodes, of any suitable animals that have been immunized with the antigen of interest. Any other suitable host cell can also be used for expressing heterologous or endogenous nucleic acid encoding an antibody, specified fragment, portion, or variant thereof. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay known to those of ordinary skill in the art (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, Del.; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684; PCT/GB91/01134; PCT/GB92/01755; PCT/GB92/002240; PCT/GB92/00883; PCT/GB93/00605; U.S. Ser. No. 08/350,260 (May 12, 1994); PCT/GB94/0 1422; PCT/GB94/02662; PCT/GB97/01835; (CAT/MRC); WO 90/14443; WO 90/14424; WO 90/14430; PCT/US94/1234; WO 92/18619; WO 96/07754 (Scripps); EP 614989 (MorphoSys); WO 95/16027 (BioInvent); WO 88/06630; WO 90/3809 (Dyax); U.S. Pat. No. 4,704,692 (Enzon); PCT/US91/02989 (Affymax); WO 89/06283; EP 371998; EP 550400; (Xoma); EP 229046; PCT/US91/07149 (Ixsys); or stochastically generated peptides or proteins—U.S. Pat. Nos. 5,723,323; 5,763,192; 5,814,476; 5,817,483; 5,824,514; 5,976,862; WO 86/05803; EP 590689 (Ixsys, now known as Applied Molecular Evolution (AME), each entirely incorporated herein by reference) or that rely upon immunization of transgenic animals (e.g., SCID mice, Nguyen et al., 41 MICROBIOL. IMMUNOL., 901-907 (1997); Sandhu et al., 16 CRIT. REV. BIOTECHNOL., 95-118 (1996); Eren et al., 93 IMMUNOL., 154-161 (1998), each entirely incorporated by reference as well as related patents and applications) that are capable of producing a repertoire of human antibodies, as known in the art and/or as described herein. Such techniques, include, but are not limited to, ribosome display (Hanes et al., 94 PROC. NATL. ACAD. SCI. USA, 4937-4942 (May, 1997); Hanes et al., 95 PROC. NATL. ACAD. SCI. USA, 14130-14135 (November, 1998); single cell antibody producing technologies (e.g., selected lymphocyte antibody method (“SLAM”) (U.S. Pat. No. 5,627,052; Wen et al., 17 J. IMMUNOL., 887-892 (1987); Babcook et al., 93 PROC. NATL. ACAD. SCI. USA, 7843-7848 (1996); gel microdroplet and flow cytometry (Powell et al., 8 BIOTECHNOL., 333-337 (1990); One Cell Systems, Cambridge, Mass.; Gray et al., 182 J. IMM. METH., 155-163 (1995); Kenny et al., 13 BIO/TECHNOL., 787-790 (1995); B-cell selection (Steenbakkers et al., 19 MOLEC. BIOL. REPORTS, 125-134 (1994); Jonak et al., Progress Biotech, Vol. 5, In Vitro Immunization in Hybridoma Technology, (Borrebaeck (Ed.), Elsevier Science Publishers B.V., Amsterdam, Netherlands (1988)).

The anti-NIgSAb can also optionally be generated by immunization of a transgenic animal (e.g., mouse, rat, hamster, non-human primate, and the like) capable of producing a repertoire of human antibodies, as described herein and/or as known in the art. Cells that produce a human anti-NIgSAb can be isolated from such animals and immortalized using suitable methods, such as the methods described herein.

Transgenic mice that can produce a repertoire of human antibodies that bind to human antigens and other foreign antigens can be produced by known methods (e.g., but not limited to, U.S. Pat. Nos: 5,770,428; 5,569,825; 5,545,806; 5,625,126; 5,625,825; 5,633,425; 5,661,016 and 5,789,650 issued to Lonberg et al.; Jakobovits et al., WO 98/50433; Jakobovits et al., WO 98/24893; Lonberg et al., WO 98/24884; Lonberg et al., WO 97/13852; Lonberg et al., WO 94/25585; Kucherlapate et al., WO 96/34096; Kucherlapate et al., EP 0463151 B1; Kucherlapate et al., EP 0710719 A1; Surani et al., U.S. Pat. No. 5,545,807; Bruggemann et al., WO 90/04036; Bruggemann et al., EP 0438474 B1; Lonberg et al., EP 0814259 A2; Lonberg et al., GB 2272440 A; Lonberg et al., 368 NATURE, 856-859 (1994); Taylor et al., 6(4) INT. IMMUNOL., 579-591 (1994); Green et al, 7 NATURE GENETICS, 13-21 (1994); Mendez et al., 15 NATURE GENETICS, 146-156 (1997); Taylor et al., 20(23) NUCLEIC ACIDS RESEARCH, 6287-6295 (1992); Tuaillon et al., 90(8) PROC. NATL. ACAD. SCI. USA, 3720-3724 (1993); Lonberg et al., 13(1) INT. REV. IMMUNOL., 65-93 (1995) and Fishwald et al., 14(7) NAT BIOTECHNOL, 845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Monospecific antibodies to native immunoglobulin are purified from mammalian antisera containing antibodies reactive against native immunoglobulin, or are prepared as monoclonal antibodies reactive with non-denatured immunoglobulin using the technique of Kohler and Milstein, 256 NATURE 495-497 (1975). Monospecific antibody as used herein is defined as a single antibody species or multiple antibody species with homogenous binding characteristics for non-denatured immunoglobulin and can be monoclonal or polyclonal. Homogenous binding as used herein refers to the ability of the antibody species to bind to a specific antigen or epitope, such as those associated with the non-denatured immunoglobulin, as described above. Native immunoglobulin-specific antibodies are raised by immunizing animals such as mice, rats, guinea pigs, rabbits, goats, horses and/or the like, with rabbits being one preferred animal, with an appropriate concentration of native immunoglobulin either with or without an immune adjuvant.

Polyclonal Antibody Preparation

Preimmune serum is collected prior to the first immunization. Each animal receives, e.g., between about 0.1 mg and about 1000 mg of native immunoglobulin associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of native immunoglobulin in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about seven days after each booster immunization or about weekly after a single immunization, the animals are bled, the serum collected, and aliquots are stored at about −20° C. This procedure may be utilized to produce polyclonal anti-NIgSAb of the present invention.

Subtractive Immunization by Using Cyclophosamide Treatment

Subtractive immunization provides a powerful alternative to standard immunization and allows for the production of truly unique antibodies. Subtractive immunization has been broadly and successfully implemented for the production of monoclonal antibodies otherwise unobtainable by standard immunization. Subtractive immunization utilizes a distinct immune tolerization approach that can substantially enhance the generation of monoclonal antibodies to desired antigens. The approach is based on tolerizing the host animal to immunodominant or otherwise undesired antigen(s) (tolerogen) that may be structurally or functionally related to the antigen of interest. Tolerization of the host animal can be achieved through one of several methods known in the art: High Zone, Neonatal, or Drug-induced tolerization. The tolerized animal is then inoculated with the desired antigen (immunogen) and antibodies generated by the subsequent immune response are screened for the desired antigenic reactivity.

By selectively killing off B-cells that have been stimulated to prolifetate in response to a foreign antigenic molecule, the cytotoxic drug cyclophosphamide can be used to manipulate the bias of the normal immune response. After cyclophosphamide treatment subsequent exposure to those molecules results in no immunological response. As a subtractive immunization technique, mice are exposed to the tolerogen followed by injections of cyclophosphamide. After the drug has been allowed to clear, the mice are exposed to the immunogen. Theoretically, the immune system should be immunologically responsive only to those molecules in the immunogen that are not found in the tolerogen. This technique of subtractive immunization may be utilized to produce the anti-NIgSAb's of the present invention.

Anti-NIgSAb of the present invention can be conveniently identified using techniques known in the art, including, but not limited to, peptide display libraries. In addition, peptide display libraries may also be used to identify NIgSBR of the present invention. This method involves the screening of large collections of peptides for individual members that are recognized or bound-to by the target molecule, which in the present invention may be native immunoglobulin or one or more epitopes thereof. Screening of peptide display libraries to find binding reagents is well known in the art. The displayed random peptide sequences can be from 3 to 5000 or more amino acids in length, frequently from 5 to 100 amino acids long, and often from about 8 to 25 amino acids long. In addition to direct chemical synthetic methods for generating peptide libraries, several recombinant DNA methods have been described. One type involves the display of random peptide sequences on the surface of a bacteriophage or cell. Each bacteriophage or cell contains the nucleotide sequence encoding the particular displayed peptide sequence. Antibody or other target molecule is immobilized on a substrate and incubated with bacteriophage or cells bearing the peptide library on their surface. After several rounds of selection by panning as described for example in Lu et al., BIO/TECHNOLOGY, 13:366-372 (1995), which is incorporated by reference herein, bacteriophage colonies are sequenced to determine the common peptide sequence recognized by the antibody. This method allows the identification of the antigen recognition sequence for the antibody or other target molecule. Such methods are described in PCT Patent Publication Nos. WO 91/18980, WO 91/19818, and WO 93/08278.

Another method well known in the art for identifying binding reagents, including NIgSBR and anti-NIgSAb of the present invention, that are specific for a particular target molecule, such as native immunoglobulin or one or more epitopes thereof, is to utilize virus, bacteriophage or host cells expressing peptide or protein molecules on their surface. In this method, DNA encoding the protein, peptide, antibody, antibody portion, antibody variant, antibody fragment, combibody, fusion protein or hybrid protein is contained within the virus, bacteriophage, host cell or other replication competent system. These molecules are expressed on the surface of the virus, bacteriophage, ribosome, host cell or other replication competent system and are selected by binding to one or more immobilized target molecule. After several rounds of selection, the DNA encoding the target-binding molecules is isolated. See PCT Patent Publication No. WO 91/17271. Other systems for generating libraries of random and specific peptides have aspects of both in vitro chemical synthesis and recombinant methods. Ribosome display libraries are also known in the art and are commercially available (Cambridge Antibody Technology, BioInvent, Affitech, Biosite). See, PCT Patent Publication Nos. WO 92/05258, WO 92/14843, and WO 96/19256. See also, U.S. Pat. Nos. 5,658,754 and 5,643,768.

Peptide display libraries, antibody fragment display libraries, hybrid protein display libraries, fusion protein display libraries, vectors, and screening kits for performing these methods are known in the art and/or are commercially available from sources such as Invitrogen (Carlsbad, Calif.), Cambridge Antibody Technologies (Cambridgeshire, UK), Phylos, Inc. (Lexington, Mass.), Dyax Corporation (Cambridge, Mass.), Morphosys (Martinsried/Munich, Germany), and Maxygen (Redwood City, Calif.). See, e.g., U.S. Pat. Nos. 4,704,692; 4,874,702; 4,939,666; 4,946,778; 5,260,203; 5,455,030; 5,518,889; 5,534,621; 5,656,730; 5,763,733; 5,767,260; 5,856,456 are assigned to Enzon; U.S. Pat. Nos. 5,223,409; 5,403,484; 5,571,698 and 5,837,500 are assigned to Dyax; U.S. Pat. Nos. 5,427,908 and 5,580,717 are assigned to Affymax; U.S. Pat. No. 5,885,793 is assigned to Cambridge Antibody Technologies; U.S. Pat. No. 5,750,373 is assigned to Genentech; U.S. Pat. Nos. 5,618,920; 5,595,898; 5,576,195; 5,698,435; 5,693,493 and 5,698,417 are assigned to Xoma; Colligan, supra; Ausubel, supra; or Sambrook, supra, each of the above patents and publications entirely incorporated herein by reference.

NIgSBR of the present invention can also be identified from diverse libraries of compounds. Such compounds may be of a wide variety of types including, but not limited to, peptides, proteins, antibodies, nucleic acids, DNA aptamers, carbohydrates, polysaccharides, fusion proteins, hybrid molecules such as peptide-nucleic acid hybrids, or any other organic or inorganic molecule or combination of molecules. Libraries of these compounds are screened for any members that bind to a target molecule. A wide variety of applicable screening methodologies are well known to those of ordinary skill in the art. Target molecules for identifying NIgSBR of the present invention from diverse libraries include, but are not limited to, native immunoglobulin and any epitope thereof. Diverse libraries that are suitable for use in identifying NIgSBR of the present invention include, but are not limited to, protein scaffold-based libraries wherein a non-immunoglobulin peptide is utilized as a framework or scaffold, upon which is built segments of variable amino acid sequences which act as the binding region for a target molecule. It is readily apparent to those of ordinary skill in the art that a wide variety of protein scaffold-based libraries are suitable for use in the methods of the present invention for identifying NIgSBR. These diverse libraries as well as methods for screening them to identify members that bind to the target molecule are well known in the art (see e.g., the protein scaffolds known as “Trinectin,” based on fibronectins, and the display technology known as “Profusion,” of Phylos, Inc., Lexington, Mass.; Affibodies based on S. aureus protein A of Affibody AB; and Anticalens based on lipocalin of Pieris Proteolab AG). Non-protein capture molecules such as DNA aptamers which bind to protein with high specificity and affinity are also used in libraries and arrays (SomaLogic). The process known in the art as “SELEX” is one methodology for the identification of these nucleic acid aptamers. These molecules are identified by one of ordinary skill in the art using these available techniques to isolate NIgSBR of the present invention.

Directed protein evolution-based libraries and screening methodologies can also be used to identify NIgSBR of the present invention. These techniques generally involve randomly inducing mutations at the genetic level, followed by selection for desired characteristics at the protein level. Directed protein evolution-based libraries and methods for making and screening them are well known in the art.

Protein arrays can also be utilized to identify NIgSBR of the present invention. Protein arrays are solid phase binding assay systems using immobilized proteins on surfaces such as glass, plastic, membranes, beads or any other surface. These arrays are used to isolate individual members from display libraries that have the selected binding characteristics. Protein arrays may be used in the methods of the present invention to select for NIgSBR and anti-NIgSAb from phage display or ribosome display libraries. Protein arrays are well known to those of ordinary skill in the art, as are methods for making and using them.

Numerous U.S. patents and published U.S. patent applications disclose the variety of libraries and methods for making and screening them to find molecules that bind to a target. Examples of these libraries and methods are disclosed in the following U.S. patents and published U.S. patent applications, and each is incorporated by reference herein. See, e.g., U.S. Pat. Nos. 6,605,449, 6,537,776 and U.S. application Ser. Nos. 2002/0146762, and 2002/0142394 assigned to Diversa Corporation; U.S. Pat. No. 5,811,238 assigned to Affymax; U.S. Pat. No. 6,489,103 assigned to Medical Research Council; U.S. application Ser. No. 2003/0186223 assigned to Dyax Corporation; U.S. Pat. Nos. 6,376,190, 6,331,398, 6,114,120, 6,110,900, 5,843,653, 5,707,796, 6,159,690, 5,696,249, 5,670,637, 5,475,096, 5,270,163, U.S. application Ser. Nos. 2003/0157487, 2003/0044818, and 2002/0102599 assigned to SELEX Techniques; U.S. Pat. Nos. 6,613,514, 6,602,986, 6,586,182, 6,579,678, 6,576,467, 6,573,098, 6,518,065, 6,506,603, 6,506,602, 6,455,253, 6,444,468, 6,436,675, 6,420,175, 6,413,774, 6,395,547, 6,372,497, 6,355,484, 6,344,356, 6,335,160, 5,323,030, 6,319,713, 6,303,344, 6,287,861, 6,297,053, 6,291,242, 6,277,638, 6,180,406, 6,165,793, 6,117,679, U.S. application Ser. Nos. 2003/0186356, and 2003/0077613 assigned to Maxygen; U.S. Pat. Nos. 6,602,685, 6,537,749, 6,436,665, 6,429,300, 6,416,950, 6,312,927 and U.S. application Ser. No. 2002/0182687 assigned to Phylos; U.S. Pat. No. 6,579,676, 5,411,861, and 5,955,264 assigned to the General Hospital Corporation; U.S. application Ser. Nos. 2002/0051998 and 2001/0051855 assigned to the California Institute of Technology; U.S. application Ser. No. 2002/0164635 assigned to Rensselaer Polytechnic Institute. U.S. application Ser. Nos. 2003/0162218, 2003/0152943, 2003/0148353, 2003/0134351, 2003/0113738, 2003/0077613, 2002/0102734, 2002/0045175, 2003/0180718 and 2003/0167128. Each of these patents and applications are incorporated by reference herein.

Anti-NIgSAb of the present invention can also be prepared using at least one anti-NIgSAb-encoding nucleic acid to provide transgenic animals or mammals, such as goats, cows, horses, sheep, and the like, that produce such antibodies in their milk. Such animals can be provided using known methods. See, e.g., but not limited to, U.S. Pat. Nos. 5,827,690, 5,849,992, 4,873,316, 5,849,992, 5,994,616, 5,565,362, 5,304,489, and the like, each of which is entirely incorporated herein by reference.

Anti-NIgSAb of the present invention can additionally be prepared using at least one anti-NIgSAb-encoding nucleic acid to provide transgenic plants and cultured plant cells (e.g., but not limited to tobacco, potato and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., 240 CURR. TOP. MICROBOL. IMMUNOL., 95-118 (1999) and references cited therein. Also, transgenic maize have been used to express mammalian proteins at commercial production levels, with biological activities equivalent to those produced in other recombinant systems or purified from natural sources. See, e.g., Hood et al., 464 ADV. EXP. MED. BIOL., 127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., 38 PLANT MOL. BIOL., 101-109 (1998) and reference cited therein. Thus, antibodies of the present invention can also be produced using transgenic plants, according to know methods. See also, e.g., Fischer et al., 30 BIOTECHNOL. APPL. BIOCHEM., 99-108 (October, 1999); Ma et al., 13 TRENDS BIOTECHNOL., 522-527 (1995); Ma et al., 109 PLANT PHYSIOL., 341-346 (1995); Whitelam et al., 22 BIOCHEM. SOC. TRANS., 940-944 (1994); Payne et al. PLANT CELL AND TISSUE CULTURE IN LIQUID SYSTEMS John Wiley & Sons, Inc. New York, N.Y. (1992); Gamborg and Phillips (eds) PLANT CELL, TISSUE AND ORGAN CULTURE; FUNDAMENTAL METHODS Springer Lab Manual, Springer-Verlag (Berlin Heidelberg New York) (1995); PLANT MOLECULAR BIOLOGY Croy (ed.) BIOS Scientific Publishers, Inc. (1993); Clark, Ed. PLANT MOLECULAR BIOLOGY: A Laboratory Manual Springer-Verlag, Berlin (1997) .and references cited therein. Each of the above references is entirely incorporated herein by reference.

The antibodies of the invention can bind native immunoglobulin proteins with a wide range of affinities (K_{D}). In a preferred embodiment, at least one anti-NIgSAb of the present invention can optionally bind native immunoglobulin protein with at least a sufficiently high affinity for use in Western blotting.

The affinity or avidity of an antibody for an antigen can be determined experimentally using any suitable method. (See, for example, Berzofsky et al., “Antibody-Antigen Interactions,” In Fundamental Immunology, Fourth Edition (W. E. Paul (Ed.), Lippincott-Raven: New York, N.Y., 1999); Janis Kuby, Immunology, (W. H. Freeman and Company: New York, N.Y., 1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffers described herein.

Nucleic Acid Molecules

Using the information provided herein, a nucleic acid molecule of the present invention encoding at least one anti-NIgSAb can be obtained using methods described herein or as known in the art.

In order to obtain nucleic acid molecules encoding the anti-NIgSAb of the present invention, the amino acid sequence of the antibody may be necessary. To accomplish this, antibody protein may be purified and partial amino acid sequence determined by automated sequenators. It is not necessary to determine the entire amino acid sequence, but the linear sequence of two regions of 6 to 8 amino acids from the protein is determined for the production of primers for PCR amplification of a partial anti-NIgSAb DNA fragment.

Once suitable amino acid sequences have been identified, the DNA sequences capable of encoding them are synthesized. Because the genetic code is degenerate, more than one codon can be used to encode a particular amino acid, and therefore, the amino acid sequence can be encoded by any of a set of degenerate DNA oligonucleotides. Only one member of the set will be identical to a given anti-NIgSAb sequence but multiple members are typically capable of hybridizing to an anti-NIgSAb encoding nucleic acid, even if the probe nucleic acid oligonucleotides has mismatches due to the degeneracy of the genetic code. The mismatched degenerate DNA oligonucleotides can typically still sufficiently hybridize to the anti-NIgSAb encoding nucleic acid to permit identification and isolation of the antibody encoding nucleic acid. DNA isolated by these methods can be used to screen DNA libraries from a variety of cell types, from invertebrate and vertebrate sources, and to isolate homologous genes.

Hybridization formats, including but not limited to solution phase, solid phase, mixed phase, or in situ hybridization assays are useful for detection of clones of interest. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook, Berger and Ausubel (herein). Labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eigth Edition by Molecular Probes, Inc. (Eugene Oreg.) (Available on CD ROM).

Nucleic acid molecules of the present invention can be in the form of RNA, such as mRNA, hnRNA, tRNA or any other form, or in the form of DNA, including, but not limited to, cDNA and genomic DNA, e.g., obtained by cloning or produced synthetically, or any combinations thereof. The DNA can be double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, e.g., but not limited to, at least one specified portion of at least one CDR, such as CDR1, CDR2 and/or CDR3 of at least one heavy chain or light chain; nucleic acid molecules comprising the coding sequence for an anti-NIgSAb or variable region; and nucleic acid molecules which comprise a nucleotide sequence substantially different from those described above but which, due to the degeneracy of the genetic code, still encode at least one anti-NIgSAb as described herein and/or as known in the art. Of course, the genetic code is well known in the art. Thus, it is routine for one skilled in the art to generate such degenerate nucleic acid variants that code for specific anti-NIgSAb of the present invention. See, e.g., Ausubel et al., supra, and such nucleic acid variants are included in the present invention.

As indicated herein, nucleic acid molecules of the present invention which comprise a nucleic acid encoding an anti-NIgSAb can include, but are not limited to, those encoding the amino acid sequence of an antibody fragment, portion or variant, by itself; the coding sequence for the entire antibody or a portion thereof; the coding sequence for an antibody, fragment, portion or variant, as well as additional sequences, such as the coding sequence of at least one signal leader or fusion peptide, with or without the aforementioned additional coding sequences, such as at least one intron, together with additional, non-coding sequences, including, but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment, portion or variant.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to an anti-NIgSAb polynucleotide sequence of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Synthetic Methods for Constructing Nucleic Acids

The isolated nucleic acids of the present invention can also be prepared by direct chemical synthesis by known methods (see, e.g., Ausubel et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA is typically most effective for sequences of about 100 or fewer bases, longer sequences can be obtained simply by the ligation of shorter sequences, via chemical or ligace mediated methods.

Recombinant Expression Cassettes

The present invention further provides recombinant expression cassettes comprising a nucleic acid of the present invention. A nucleic acid sequence of the present invention, for example a cDNA or a genomic sequence encoding an antibody of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

Vectors and Host Cells

The present invention also relates to vectors that include isolated nucleic acid molecules of the present invention, host cells that are genetically engineered with the recombinant vectors, and the production of at least one anti-NIgSAb by recombinant techniques, as is well known in the art. See, e.g., Sambrook et al., supra; Ausubel et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome-binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably, but optionally, include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636 and 5,179,017; ampicillin, neomycin (G418), mycophenolic acid, or glutamine synthetase (GS, U.S. Pat. Nos. 5,122,464; 5,770,359 and 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention. Alternatively, nucleic acids of the present invention can be expressed in a host cell by turning on (by manipulation) expression in a host cell that contains endogenous DNA encoding an antibody of the present invention. Such methods are well known in the art, e.g., as described in U.S. Pat. Nos. 5,580,734; 5,641,670; 5,733,746 and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). In one embodiment, host cells include cells of lymphoid origin such as myeloma and lymphoma cells.

Expression vectors for these cells can include one or more of the following expression control sequences, such as, but not limited to, an origin of replication; a promoter (e.g., late or early SV40 promoters, the CMV promoter (U.S. Pat. Nos. 5,168,062 and 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague et al., 45 J. VIROL., 773-781 (1983)). Additionally, gene sequences to control replication in the host cell can be incorporated into the vector, as known in the art.

Purification of an Antibody

An anti-NIgSAb can be recovered and purified from serum, or hybridoma or recombinant cell cultures by well-known methods including, but not limited to, protein A or protein G purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, (John Wiley & Sons, New York, N.Y., 1997-2001), e.g., Chapters 1, 4, 6, 8, 9, 10, each entirely incorporated herein by reference. In addition to many of the other references noted herein, a variety of purification and associated protein folding and re-folding methods are well known in the art and can be applied to antibody or other NIgSBR molecule purifications, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana Bioseparation of Proteins, Academic Press, Inc. (1997); Bollag et al. Protein Methods, 2nd Edition Wiley-Liss, NY (1996); Walker The Protein Protocols Handbook Humana Press, NJ (1996); Harris and Angal Protein Purification Applications: A Practical Approach IRL Press at Oxford, Oxford, England (1990); Scopes Protein Purification: Principles and Practice 3rd Edition Springer Verlag, NY (1993); Janson and Ryden Protein Purification: Principles, High Resolution Methods and Applications, Second Edition Wiley-VCH, NY (1998); and Walker Protein Protocols on CD-ROM Humana Press, NJ (1998); and the references cited therein.

NIgSBR and anti-NIgSAb of the present invention can include any of: naturally purified products, products of chemical synthetic procedures, and products produced by recombinant techniques from a prokaryotic or eukaryotic host, including, for example, bacteria, fungi, yeast, plant, insect, non-mammalian, and mammalian cells. Depending upon the host employed in a recombinant production procedure, the antibody of the present invention can be glycosylated or can be non-glycosylated, with glycosylated being preferred in certain embodiments. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

The polyclonal anti-NIgSAb of the present invention can be treated to remove any antibodies that bind to denatured immunoglobulin. This treatment can be done by contacting the polyclonal anti-NIgSAb with denatured immunoglobulin, and then removing the antibodies that are bound to the denatured immunoglobulin. This removal may be achieved using denatured immunoglobulin that is attached to an insoluble substrate, for example. Any of the antibody that binds to the denatured immunoglobulin attached to the substrate can be separated from the remaining antibodies supply by collecting the unbound antibodies or by removing the insoluble substrate with the denatured immunoglobulin attached along with the antibodies bound to the denatured immunoglobulin. It is readily apparent to those of ordinary skill in the art that the insoluble substrate can be a bead, plate, well, tube, sheet or any of a wide variety of shapes, sizes or materials.

Preparation of NIgSBR's

Any NIgSBR that is not an antibody may be identified, isolated and characterized generally as described herein. It is readily apparent to those of ordinary skill in the art that any particular non-antibody NIgSBR will generally be amenable to standard methods and procedures known in the art to be suitable or appropriate for the particular class of NIgSBR compound or molecule. One of ordinary skill in the art will readily appreciate and be able to select the appropriate or suitable methods for isolating, purifying, formulating, handling, etc., the particular NIgSBR of interest without undue experimentation. As noted, for protein or related NIgSBR, the following references provide considerable detail on such procedures: Scopes, Protein Purification, Springer-Verlag, N.Y. (1982); Deutscher, Methods in Enzymology Vol. 182: Guide to Protein Purification, Academic Press, Inc. N.Y. (1990); Sandana Bioseparation of Proteins, Academic Press, Inc. (1997); Bollag et al. Protein Methods, 2nd Edition Wiley-Liss, NY (1996); Walker The Protein Protocols Handbook Humana Press, NJ (1996); Harris and Angal Protein Purification Applications: A Practical Approach IRL Press at Oxford, Oxford, England (1990); Scopes Protein Purification: Principles and Practice 3rd Edition Springer Verlag, NY (1993); Janson and Ryden Protein Purification: Principles, High Resolution Methods and Applications, Second Edition Wiley-VCH, NY (1998); and Walker Protein Protocols on CD-ROM Humana Press, NJ (1998). For Nucleic acid manipulations, Sambrook and Ausubel provide detailed procedures for isolating, purifying, formulating and handling nucleic acids (as well as proteins and certain small molecules). For organic synthesis techniques and methods for isolating, purifying, formulating and handling resulting molecules, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Further details regarding the isolating, purifying, formulating and handling of many compounds is found in the 2004 Sigma catalogue (USA) or 2004 Aldrich catalogue (Milwaukee, Wis., USA).

Diagnostic Methods

The present invention also provides anti-NIgSAb, detectably labeled, as described herein, for use in research, therapeutic or diagnostic methods.

Anti-NIgSAb of the present invention are useful for a wide variety of procedures such as immunoassays which detect or quantitate other antigens or antibodies in a sample or on a substrate. An immunoassay for detecting antibodies typically comprises incubating a sample in the presence of a detectably labeled anti-NIgSAb of the present invention, and detecting the labeled antibody which is bound in a sample. Various clinical assay procedures are well known in the art, e.g., as described in Immunoassays for the 80's, (Eds. A. Voller et al., University Park, 1981) and in Paul (ed.) FUNDAMENTAL IMMUNOLOGY, FOURTH EDITION, Lippincott-Raven, NY, N.Y. (1999).

Thus, an anti-NIgSAb, a NIgSBR or any other antibody molecule used in the assay, can be added to nitrocellulose, or another solid support which is capable of immobilizing cells, cell contents or proteins. The support can then be washed with suitable buffers and other desired reagents, followed by treatment with the detectably labeled anti-NIgSAb or detectably labeled NIgSBR. The solid phase support can then be washed with the buffer a second time to remove unbound detectably labeled anti-NIgSAb or unbound detectably labeled NIgSBR. The amount of bound label on the solid support can then be detected or quantified by known method steps.

“Solid phase support” or “carrier” includes any support capable of binding peptide, protein, antigen or antibody. Well-known supports or carriers include glass, polystyrene, polypropylene, polyethylene, dextran, nylon, amylases, natural and modified celluloses, nitrocellulose, polyacrylamides, agaroses, and magnetite. The nature of the carrier can be either soluble to some extent or insoluble for the purposes of the present invention. The support material can have virtually any possible structural configuration so long as the coupled molecule is capable of binding to antigen or antibody. Thus, the support configuration can be spherical, as in a bead, or cylindrical, as in the inside surface of a test tube, or the external surface of a rod. Alternatively, the surface can be flat such as a sheet, culture dish, test strip, etc. Preferred supports include polystyrene beads and wells of a plate. It is readily apparent to those skilled in the art that there are many other suitable carriers for binding antibody, peptide or antigen, or can ascertain the same by routine experimentation.

Well known method steps can determine binding activity of a given lot of anti-NIgSAb or NIgSBR. Those skilled in the art can determine operative and optimal assay conditions by routine experimentation using well known methods in additional to those disclosed herein.

Detectably labeling an anti-NIgSAb or NIgSBR can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the anti-NIgSAb of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

By radioactively labeling the anti-NIgSAb or NIgSBR, it is possible to detect non-denatured immunoglobulin through the use of a radioimmunoassay (RIA) (see, for example, Work, et al., Laboratory Techniques and Biochemistry in Molecular Biology, North Holland Publishing Company, N.Y. (1978). The radio-active isotope can be detected by such means as the use of a gamma counter or a scintillation counter or by autoradiography. Isotopes which are particularly useful for the purpose of the present invention are: ³H, ¹²⁵I, ¹³¹I, ³⁵S, ¹⁴C, and ¹²⁵I.

It is also possible to label the anti-NIgSAb or NIgSBR with a fluorescent compound. When the fluorescent labeled agent is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. See also, Haugland, HAND BOOK OF FLOURESCENT PROBES AND RESEARCH PRODUCTS, NINTH EDITION Molecular Probes, Inc., Eugene Oreg. (2003).

The anti-NIgSAb or NIgSBR can also be detectably labeled using fluorescence-emitting metals such as ¹⁵²Eu, or others of the lanthanide series. These metals can be attached to the anti-NIgSAb using such metal chelating groups as diethylenetriaminepentaacetic acid (DTPA) or ethylenediamine-tetraacetic acid (EDTA).

The anti-NIgSAb or NIgSBR also can be detectably labeled by coupling to a chemiluminescent compound. The presence of the chemiluminescently labeled antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the anti-NIgSAb or NIgSBR of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the anti-NIgSAb or NIgSBR can be accomplished, e.g., by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

In situ detection can be accomplished, e.g., by removing a histological specimen from a patient, and providing the combination of detectably labeled anti-NIgSAb or NIgSBR of the present invention to such a specimen. The detectably labeled anti-NIgSAb or NIgSBR is preferably provided by applying or by overlaying the detectably labeled anti-NIgSAb or NIgSBR to a biological sample. Through the use of such a procedure, it is possible to determine not only the presence of native immunoglobulin but also the distribution of native immunoglobulin in the examined tissue. Using the present invention, those of ordinary skill will readily perceive that any of a wide variety of histological methods (such as staining procedures) can be modified in order to achieve such in situ detection.

The anti-NIgSAb or NIgSBR of the present invention can be adapted for utilization in an immunometric assay, also known as a “two-site” or “sandwich” assay. In a typical immunometric assay, a quantity of unlabeled antibody (or fragment of an antibody) is bound to a solid support that is insoluble in the fluid being tested and a quantity of detectably labeled anti-NIgSAb or NIgSBR is added to permit detection and/or quantitation of the ternary complex formed between solid-phase antibody, antigen, and detectably labeled agent.

Typical, immunometric assays include “forward” assays in which the antibody bound to the solid phase is first contacted with the sample being tested to extract the antigen from the sample by formation of a binary solid phase antibody-antibody complex. After a suitable incubation period, the solid support is washed to remove the residue of the fluid sample, including unreacted antigen, if any, and then contacted with the solution containing a known quantity of labeled antibody (which functions as a “reporter molecule”). After a second incubation period to permit the labeled antibody to complex with the antigen bound to the solid support through the unlabeled antibody, the solid support is washed a second time to remove the unreacted labeled antibody. This type of forward sandwich assay can be a simple “yes/no” assay to determine whether antigen is present or can be made quantitative by comparing the measure of labeled antibody with that obtained for a standard sample containing known quantities of non-denatured immunoglobulin. Such “two-site” or “sandwich” assays are described by Wide (Radioimmune Assay Method, (Ed. Kirkham, Livingstone, Edinburgh (1970) 199-206).

Other types of “sandwich” assays, which can also be useful with native immunoglobulin, are the so-called “simultaneous” and “reverse” assays. A simultaneous assay involves a single incubation step wherein the antibody bound to the solid support, antigen, and labeled antibody and other desired reagents are added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The presence of labeled antibody associated with a solid support is then determined as in the “simultaneous” and “forward” assays. In one embodiment, a combination of anti-NIgSAb and/or NIgSBR of the present invention specific for the same or separate epitopes can be used to construct a sensitive multi-site immunoradiometric assay.

Kits or articles of manufacture containing the anti-NIgSAb or NIgSBR of the present invention may be prepared. Such kits or articles of manufacture are used in the performance of research, therapeutic, or diagnostic procedures.

Typically in the methods described herein the anti-NIgSAb and NIgSBR are interchangeable and can be substituted one for the other, or used in combination one with the other, including where they are detectably labeled.

The following examples illustrate the present invention without, however, limiting the same thereto. One of skill will recognize a variety of essentially equivalent parameters and materials that can be substituted in accordance with the present invention.

### EXAMPLE 1

Production of Antibody

Immunization and Fusion Methods

Lou/M rats were immunized with mouse serum immunoglobulin. Balb/c mice were immunized with rabbit serum immunoglobulin. Sera titer were tested after three immunizations. Spleens from animals with high serum titer were fused with a mouse myeloma fusion partner, SP2/O.

Fusion Protocol

A. Media Preparation

1) IMDM basic medium:

2) 20% FBS Complete medium:
500 ml IMDM basic medium+100 ml FBS+6.5 ml P/S (stock solution concentration: 10,000 units/ml penicillin; 100,000 units/ml streptomycin)+6.5 ml glutamine (stock solution concentration: 200 μM).

3) Supplements:
100× HAT
50× HES (Hybridoma Enhancing Supplement)

4) Fusion medium:
500 ml complete medium
5 ml 100× HAT
10 ml 50× HES

5) 50% PEG (Sigma, MW 1500)

B. Preparation of the Myeloma Cells

1) Prior to the fusion, the myeloma cell line was cultured in at least 10-20% FBS IMDM medium for three to four days. The myeloma were kept in log phase of growth prior to fusing the cells.

2) Using a light microscope, the myeloma cells were checked for growth and viability.

3) The myeloma cells were dislodged gently from the surface of the flasks and transferred into 50 ml conical tubes.

4) Myeloma cells were centrifuged at 1100 rpm for five minutes.

5) The supernatants were discarded and the cell pellet re-suspended in plain IMDM medium.

6) The myeloma cells were centrifuged at 1100 rpm for five minutes.

7) The supernatant was discarded and the cell pellet re-suspended in basic IMDM medium.

8) The washing was repeated once and a cell count was performed.

C. Preparation of spleen cells was done as follows:

1) The animal was anesthetized and an exsanguination was performed via cardiac puncture to collect as much blood as possible, followed by cervical dislocation.

2) The animal was bathed in 75% ethanol for two to three minutes.

3) The animal was placed on its right side to expose the left ventral area and pinned down on Styrofoam rack that was sterilized with 75% ethanol.

4) The skin of the left leg was cut with a pair of scissors into a V-shape. The incision was enlarged by pulling the skin away from the cut, thereby exposing the abdominal peritoneum. The spleen was visible as a reddish dark mass right underneath the peritoneum of the left ventral abdomen.

5) The peritoneum was cut open with a small pair of scissors to expose the spleen.

6) The spleen was dissected and the adhesions teased away to remove the spleen. The spleen was placed on a pre-wetted nylon screen cell strainer placed on top of a 50 ml conical tube. The spleen was cut into small pieces with a small pair of curved scissors.

7) The spleen pieces were meshed against the cell strainer with a 1 cc syringe plunger and homogenized with a milling action. The spleen cells were rinsed into the 50 ml conical tube with basic IMDM medium (penicillin/streptomycin optional). The spleen cells were continuously homogenized and rinsed until all spleen cells were isolated.

8) The spleen cells were centrifuged at 1100 rpm for five minutes. The supernatant was discarded and the pellet re-suspended. 40 ml IMDM was added to the cells and the centrifugation was repeated as before. This was the FIRST WASH.

9) The washed spleen cell pellet was re-suspended with IMDM medium and a cell count was performed.

D. Fusion of the spleen cells and myeloma was done as follows:

1) The spleen cells and myeloma cells were combined together in a 2-5:1 spleen cell to myeloma cell ratio (2:1 or 3:1 is preferred).

2) The cell mixture was centrifuged at 1500 rpm for seven minutes.

3) The supernatant was aspirated off using a glass pastuer pipet, ensuring no moisture was found on the sides of the 50 ml conical tube.

4) The pellet was gently dislodged by hitting the side of the tube against a styrofoam rack.

5) 50% PEG (pre-warmed to 37° C.) was added dropwise over one minute while stirring occasionally.

| | |
|---|---|
| Mouse or hamster fusion | 0.8-1.0 mls PEG |
| Rat fusion | 0.9-1.2 mls PEG |

6) The cell/50% PEG suspension was allowed to react

| | |
|---|---|
| Mouse or Hamster fusion: | 1 minute 15 seconds |
| Rat fusion: | 1 minute 30 seconds |

7) 15 mls of pre-warmed plain IMDM medium were gradually added over five minutes:

a) one ml added in first one minute with occasionally mixing.

b) 2 ml medium added over second one minute with occasionally mixing.

c) the remaining medium was added in the next two to three minutes while mixing occasionally.

8) The cell suspension was incubated in 37° C. water bath for two to five minutes.

9) The cells were centrifuged at 1000 rpm for five minutes.

10) The supernatants were discarded and the pellet gently re-suspended. The cells were then carefully transferred into the proper volume of the fusion medium (HAT medium).

11) After 30 minutes to one hour, the hybridoma culture was plated out at 200 μper well with wide orifice tips into 96-well flat-bottom tissue culture plates.

12) The mixture was cultured at 37° C., 7% CO2 incubator with 100% humidity for three to four days. ⅔ of the supernatant was aspirated off from all wells and then carefully added back 140-180 μl/well HT medium with wide orifice tips to prevent breaking up the cell colonies.

13) The hybridoma supernatant was screened after Days 9 to 10.

Characterization of Antibody's Preferential Reactivity to Native Immunoglobulin.

Screening: All clones in 96 well plates were screened by ELISA as follows:

1) The ELISA plate was coated with 100 ul/well of mouse serum IgG in PBS at concentration of 2 ug/ml. The plate was sealed and incubated at 4° C. overnight.

2) The wells were aspirated and washed three times with greater than 300 ul/well Wash Buffer. The plate was inverted and blotted on absorbent paper to remove any residual buffer.

3) The wells were blocked with 200 ul/well of Assay Diluent and incubated at room temperature for one hour.

4) Step two was repeated.

5) 100 ul/well of the TCS samples was added to the wells. The plate was sealed and incubated at room temperature for two hours.

6) Step 2 was repeated for a total of five washes.

7) 100 ul/well of HRP-conjugated mouse anti-rat antibody was added to the Assay Diluent at a concentration of 1:2000. The plate was sealed and incubated at room temperature for one hour.

8) Step 2 was repeated for a total of five washes.

9) The ABTS substrate was thawed within 20 minutes of use and 11 ul of 30% H2O2 was added per 11 ml of substrate. 100 ul/well of ABTS substrate solution was added to each well. The plate was incubated at room temperature for 10 to 20 minutes.

10) The plate was read at 405 nm.

All positive clones were selected and the following tests were done:
1) Specificity test: ELISA plates were coated with 100 ul/well of different mouse isotypes at 2 ug/ml in PBS. Mouse IgG1. 2a, 2b, G3, IgA and IgM were added to row A, B, C, D, E, F and G. Samples were added to column 1, 2, 3, 4, etc. The other assay steps were done as described for the above screening protocol.
2) Isotype test: ELISA plates were coated with 100 ul/well of different anti-mouse isotype mAbs at 2 ug/ml in PBS. Anti-mouse IgG1. 2a, 2b, G3, IgA and IgM were added to row A, B, C, D, E, F and G. Samples were added to column 1, 2, 3, 4, etc. The other assay steps were done as described for the above screening protocol.
3) Western blot/immunoblotting of immunoglobulin was done as follows:
   1) Serum IgG (native and denatured) samples were prepared to run an SDS-PAGE gel.
   2) The samples were transferred from the gel onto Immobilon-P membranes following instructions provided by the transfer system manufacturer for best protein transfer results.
   3) The blot was incubated with TCS sample in the Antibody Binding Buffer overnight at 4° C.
   4) After the overnight incubation of the membrane with the sample, the blot was washed five times for five minutes in TBST.
   5) The blot was incubated with anti-Ig HRP-conjugated at 1:2000 dilution for one hour at room temperature.
   6) After incubation of the anti-NIgSAb, the blot was washed five times for five minutes in TBST.
   7) The blot was developed following the Pierce Chemiluminescence HRP substrate instruction provided by the manufacturer.
   8) The blot was exposed to photographic film for the appropriate time period. For best results, the exposure should last between one minute and five minutes to visualize the chemiluminescence signal corresponding to the specific antibody-antigen reaction. The clones that reacted with native immoglobulin, but not denatured immunoglobulin, were scaled up, purified and conjugated to HRP anti-NIgSAb, and then the immunoprecipitation/Western blot (IP/WB) tests were run:
4) IP/WB was done as follows:

Step I: Cell Lysate Preparation was Done as Follows:

1. Harvest Jurkat cell approximately 10⁷ cells.

2. The cells were washed with about 10 ml of PBS in a conical tube and centrifuged at 400×g for ten minutes.

3. The supernatant was discarded and Step 2 was repeated.

4. After the second wash, the supernatant was completely discarded and the cell pellet was re-suspended in 1 ml of cold Lysis Buffer containing 1× Protease Inhibitor Cocktail (final concentration of 107 cells/ml). The tube was gently vortexed.

5. The tube was placed on ice for 30 minutes, with occasional mixing.

6. The cell lysate was centrifuged at 10,000×g for 15 minutes at 4° C.

7. The supernatant was careful collected without disturbing the pellet and transferred to a clean tube. The cell lysate can be frozen at this point for long-term storage at −80° C. The pellet was discarded.

Step II: Cell Lysate Preclearing was Done as Follows:

1. 50 μl of anti-immunoglobulin bead slurry was transferred to a test-tube and 450 μl cold Lysis Buffer was added. The mixture was centrifuged at 10000×g for 60 seconds and the Lysis Buffer was removed. The wash was repeated with 500 μl of cold Lysis Buffer and the beads were re-suspended in 50 μl of cold Lysis Buffer.

2. The 50 μl of anti-immunoglobulin bead slurry and 500 μl of Cell Lysate were added to a test-tube and incubated on ice for 60 minutes.

3. The mixture was centrifuged at 10000×g for ten minutes at 4° C. and the supernatant was transferred to a fresh test-tube. If any bead was transferred, the supernatant must be re-centrifuged and transferred to another fresh test-tube.

Step III: Immunoprecipitation was Done as Follows:

1. 5 μg of anti-human caspase-7 antibody was added to the test-tube containing the cold precleared lysate.

2. The mixture was incubated at 4° C. for one hour.

3. 50 μl of anti-immunoglobulin bead slurry in pre-chilled Lysis Buffer was added to the mixture (prepared as instructed in Preclearing Step 1 above).

4. The mixture was incubated for one hour at 4° C. on a rocking platform or a rotator.

5. The test-tube was centrifuged at 10000×g for 60 seconds at 4° C.

6. The supernatant was carefully and completely removed and the beads were washed three times with 500 μl of Lysis Buffer. To minimize background, care was given to remove the supernatant completely in these washes.

7. After the last wash, the supernatant was aspirated and 50 μl of sample buffer was added to the bead pellet which was mixed and heated to 100° C. for ten minutes.

8. The mixture was centrifuged at 10,000×g for five minutes, then the supernatant was collected and loaded onto an SDS-PAGE gel. Supernatant samples can be collected and kept frozen at this point if the gel is to be run later.

9. Follow manufacturer's instructions for SDS-PAGE.

10. The samples were transferred from the SDS-PAGE gel onto Immobilon-P membranes following instructions provided by the transfer system manufacturer.

11. The blot was incubated with anti-human caspase-7 mAb (primary antibody) at 2 ug/ml in the Antibody Binding Buffer overnight at 4° C.

12. After the overnight incubation of the membrane with the primary antibody, the blot was washed five times for five minutes in TBST.

13. The blot was incubated with HRP-conjugated 2nd step mAb (anti-NIgSAb) at 1:250 dilution for one hour at room temperature.

14. After incubation of the secondary antibody, the blot was washed five times for five minutes in TBST.

15. The blot was developed following the Pierce Chemiluminescence HRP substrate instructions.

16. The blot was exposed to photographic film for the appropriate time period. For best results, expose for one minute and five minutes to visualize the chemiluminescence signal corresponding to the specific antibody-antigen reaction.

The methods of the present invention selectively enriched for antibody that binds with high preference to native immunoglobulin for an optimal signal while reducing the background noise attributable to binding to the denatured light and heavy chain molecules carried over from the immunoprecipitation steps.

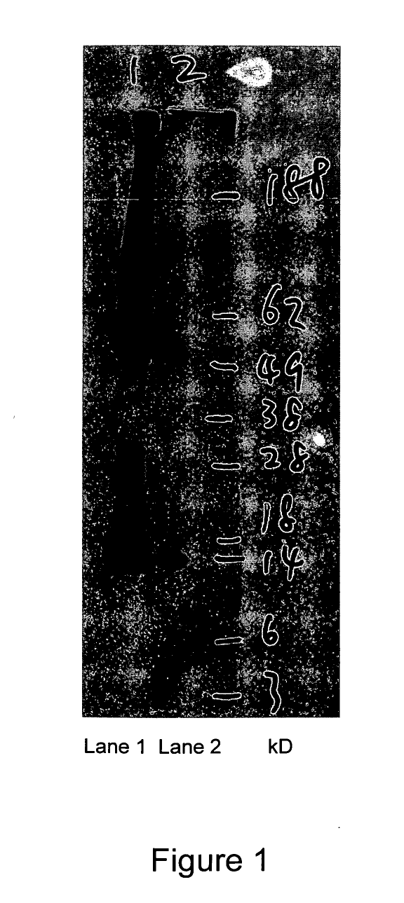

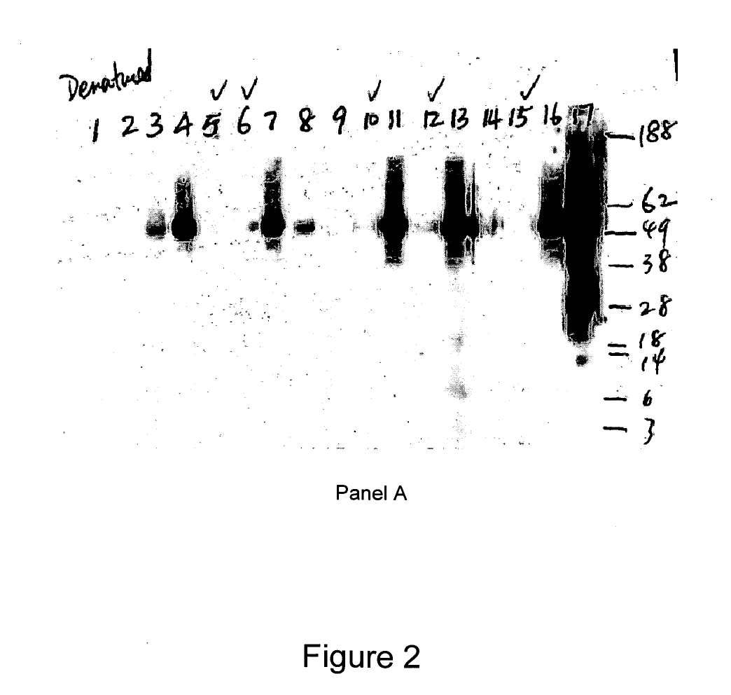

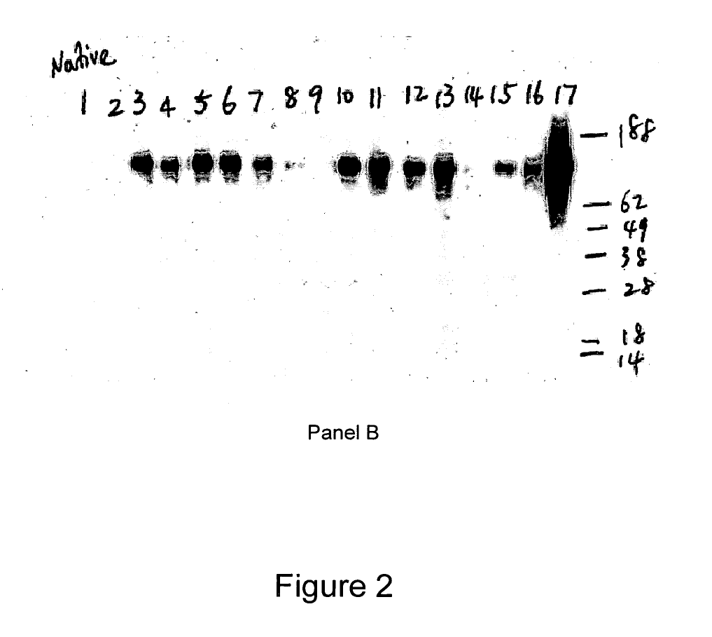

The anti-NIgSAb and NIgSBR of the present invention, as exemplified by monoclonal antibody produced by clone eB144 to specifically bind to native immunoglobulin on IP/Western blots is shown in FIGS. 1, 2 and 3, where the figures demonstrate the substantial advantage of the anti-NIgSAb and NIgSBR of the present invention to selectively react with the native immunoglobulin only, as compared with a conventional polyclonal antibody which reacts with both the heavy and light chain of denatured immunoglobulin molecules. As shown, assigning a value of 100% binding to native immunoglobulin, approximately 0% binding to denatured immunoglobulin molecules was detected with the anti-NIgSAb.

The results of an immunoblot using detectably labeled anti-NIgSAb of the present invention is shown in FIG. 1. 100 ng recombinant human IL-2 was immunoprecipited with rabbit anti-human IL-2 polyclonal antibody. SDS-PAGE was loaded 20 ng per well and transferred to a membrane for Western blotting. Lane 1 was incubated with conventional labeled polyclonal antibody (Amersham donkey anti-rabbit Igs, 1:5000)—extra bands on the lane show denatured immunoglobulin light and heavy chain contaminants. Lane 2 is incubated with detectably labeled monoclonal rat anti-rabbit Ig supernatant prepared according to the methods of the present invention.

The selection process for the lack of reaction of the antibodies produced by the methods of the present invention with the denatured rabbit immunoglobulins is shown. Each lane was probed using a different antibody. The corresponding lanes of each panel were probed with the same antibody. The production of antibodies that preferentially bind native antibodies is shown. Lanes 5, 6, 10, 12, 15 each show anti-NIgSAb's showing little or no antibody reactivity to denatured rabbit immunoglobulin by Western blotting. Lanes 4, 7, 8, 11, 13, 14, 16, and 17 each reacted with both native and denatured immunoglobulins.

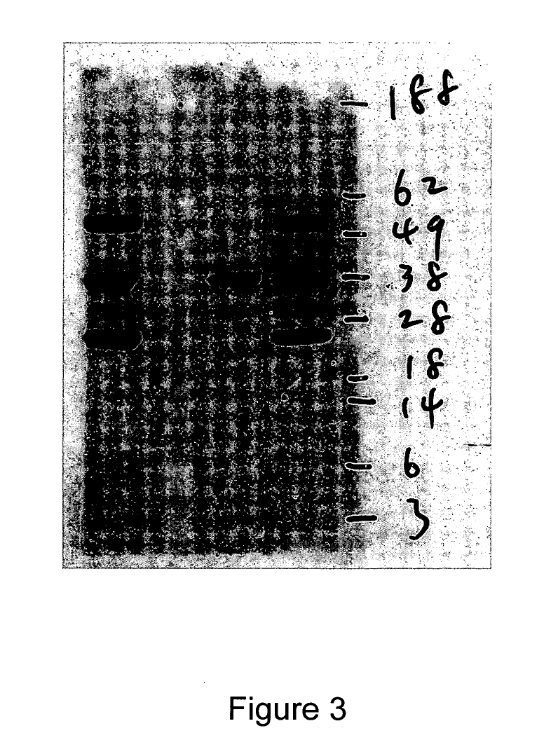

FIG. 3 shows the specificity of the anti-NIgSAb of the present invention for non-denatured immunoglobulin. JURKAT cell lysate (0.5 ml of 1×10e7 cells/ml) was immunoprecipited with mouse anti-human Caspase 7 (5 μg). SDS-PAGE was loaded 10 l per well (1×10⁶ cells) and transferred to a membrane for Western blotting. Lane 1 was incubated with labeled conventional polyclonal antibody (Jackson polyclonal anti-mouse Igs, 1:5000)—extra bands on the lane show light and heavy chain contaminants. Lane 2 was incubated with detectably labeled anti-NIgSAb (anti-mouse Ig) of the present invention (1:300). Lane 3 is a re-blot of Lane 2 using the labeled conventional polyclonal antibody (Jackson polyclonal anti-mouse Igs, 1:5000) that was used for Lane 1. The appearance of the extra bands on Lane 3 show light and heavy chain contaminants, confirming that the detectably labeled anti-NIgSAb (anti-mouse Ig) of the present invention (1:300) selectively and preferentially binds the native Ig over the contaminating denatured Ig.

### EXAMPLE 2

Subtractive Immunization to Prepare Polyclonal Anti-NIGSAB

Procedure:

Day 1: Inject 6 mice (i.p.) with tolerogens (denatured immunoglobulin) which are not desired for the final antibody production (25-50 mg) using complete adjuvant. Ten minutes later inject 100 mg/kg body weight of cyclophosphamide (SIGMA) in sterile phosphate buffered saline. Make a 2 mg/ml of cyclophosphamide solution for this purpose.

Day 2: Inject the cyclophosphamide again (100 mg/kg body weight).

Day 3: Repeat injection of cyclophosphamide.

Day 7: Bleed mice and do an antibody titer via ELISA.

Day 14: Inject 6 mice (i.p.) with tolerogen (25-50 mg) which are not desired for final antibody production using incomplete adjuvant. Ten minutes later inject 100 mg/kg body weight of cyclophosphamide in sterile phosphate buffered saline.

Day 15: Inject the cyclophosphamide again (100 mg/kg body weight).

Day 16: Repeat injection of cyclophosphamide.

Day 21: Bleed mice and do an antibody titer. If no antibody titer is acquired proceed to the next step. If there still exist an antibody titer repeat injections with the tolerogens and cyclophosphamide.

Day 28: Immunize with the desired immunogen (native immunoglobulin) using complete adjuvant.

Day 38: Bleed mice and do antibody titer assay.

Day 42: Repeat immunization with immunogen using incomplete adjuvant.

Day 46: Bleed mice for antibody titer. This antibody represents polyclonal anti-NIgSAb. If desired antibody titer (about 1/10³ or greater) is obtained proceed with cell fusion the next day if monoclonal antibody is to be produced for the animals. If desired titer is not obtained repeat immunogen in adjuvant injections every two weeks until desired titer is obtained.

### EXAMPLE 3

Cloning and Expression of Anti-NIGSAB in Mammalian Cells

A typical mammalian expression vector contains at least one promoter element, which mediates the initiation of transcription of mRNA, the antibody coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (±), pcDNA/Zeo (±) or pcDNA3.1/Hygro (±) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded antibody. The DHFR (dihydrofolate reductase) marker is useful to develop cell lines that carry several hundred or even several thousand copies of the gene of interest. Another useful selection marker is the enzyme glutamine synthase (GS) (Murphy et al., 227 BIOCHEM. J., 277-279 (1991); and Bebbington et al., 10 BIO/TECHNOLOGY, 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of antibodies.

The expression vectors pC1 and pC4 contain the strong promoter (LTR) of the Rous Sarcoma Virus (Cullen et al., 5 MOLEC. CELL. BIOL., 438-447 (1985)) plus a fragment of the CMV-enhancer (Boshart et al., 41 CELL, 521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### EXAMPLE 4

Generation of Monoclonal Antibodies Reactive with Non-Denatured Immunoglobulin

Mice can be used to generate monoclonal antibodies that can be used in the methods of the present invention.

Immunization

One or more immunization schedules can be used to generate the anti-NIgSAb hybridomas. The first several fusions can be performed after the following exemplary immunization protocol, but other similar known protocols can be used. Several fourteen to twenty week-old female and/or surgically castrated male mice are immunized IP and/or ID with 1 to 1000 μg of the desired immunoglobulin protein emulsified with an equal volume of TITERMAX or complete Freund's adjuvant in a final volume of 100 to 400 μL (e.g., 200). Each mouse can also optionally receive 1 to 10 μg in 100 μL physiological saline at each of two SQ sites. The mice can then be immunized 1 to 7, 5 to 12, 10 to 18, 17 to 25 and/or 21 to 34 days later IP (1 to 400 μg) and SQ (1 to 400 μg×2) with the immunoglobulin protein emulsified with an equal volume of TITERMAX or incomplete Freund's adjuvant. Mice can be bled 12 to 25 and 25 to 40 days later by retro-orbital puncture without anti-coagulant. The blood is then allowed to clot at room temperature for one hour and the serum is collected and titered using an anti-immunoglobulin EIA assay according to known methods. Fusions are performed when repeated injections do not cause titers to increase. At that time, the mice can be given a final IV booster injection of 1 to 400 μg of the immunoglobulin protein diluted in 100 μL physiological saline. Three days later, the mice can be euthanized by cervical dislocation and the spleens removed aseptically and immersed in 10 mL of cold phosphate buffered saline (PBS) containing 100 U/mL penicillin, 100 μg/mL streptomycin, and 0.25 μg/mL amphotericin B (PSA). The splenocytes are harvested by sterilely perfusing the spleen with PSA-PBS. The cells are washed once in cold PSA-PBS, counted using Trypan blue dye exclusion and re-suspended in RPMI 1640 media containing 25 mM Hepes.

Cell Fusion

Fusion can be carried out at a 1:1 to 1:10 ratio of murine myeloma cells to viable spleen cells according to known methods, e.g., as known in the art. As a non-limiting example, spleen cells and myeloma cells can be pelleted together. The pellet can then be slowly re-suspended, over thirty seconds, in 1 mL of 50% (w/v) PEG/PBS solution (PEG molecular weight 1,450, Sigma) at 37° C. The fusion can then be stopped by slowly adding 10.5 mL of RPMI 1640 medium containing 25 mM Hepes (37° C.) over one minute. The fused cells are centrifuged for five minutes at 500 to 1500 rpm. The cells are then re-suspended in HAT medium (RPMI 1640 medium containing 25 mM Hepes, 10% Fetal Clone I serum (Hyclone), 1 mM sodium pyruvate, 4 mM L-glutamine, 10 μg/mL gentamicin, 2.5% Origen culturing supplement (Fisher), 10% 653-conditioned RPMI 1640/Hepes media, 50 μM 2-mercaptoethanol, 100 μM hypoxanthine, 0.4 μM aminopterin, and 16 μM thymidine) and then plated at 200 μL/well in fifteen 96-well flat bottom tissue culture plates. The plates are then placed in a humidified 37° C. incubator containing 5% CO₂ and 95% air for seven to ten days.

Detection of Anti-NIgSAb in Mouse Serum

Solid phase EIA's can be used to screen mouse sera for human IgG antibodies specific for native immunoglobulin. Briefly, plates can be coated with immunoglobulin at 2 μg/mL in PBS overnight. After washing in 0.15M saline containing 0.02% (v/v) Tween 20, the wells can be blocked with 1% (w/v) BSA in PBS, 200 μL/well for one hour at room temperature. Plates are used immediately or frozen at −20° C. for future use. Mouse serum dilutions are incubated on the immunoglobulin-coated plates at 50 μL/well at room temperature for one hour. The plates are washed and then probed with 50 μL/well HRP-labeled goat anti-human IgG, Fc-specific diluted 1:30,000 in 1% BSA-PBS for one hour at room temperature. The plates can again be washed and 100 μL/well of the citrate-phosphate substrate solution (0.1M citric acid and 0.2M sodium phosphate, 0.01% H₂O₂ and 1 mg/mL OPD) is added for fifteen minutes at room temperature. Stop solution (4N sulfuric acid) is then added at 25 μL/well and the OD's are read at 490 nm via an automated plate spectrophotometer.

Detection of Immunoglobulins in Hybridoma Supernates

Growth positive hybridomas secreting immunoglobulins can be detected using a suitable EIA. Briefly, 96 well pop-out plates (VWR, 610744) can be coated with 10 μg/mL goat anti-mouse IgG Fc in sodium carbonate buffer overnight at 4° C. The plates are washed and blocked with 1% BSA-PBS for one hour at 37° C. and used immediately or frozen at −20° C. Undiluted hybridoma supernatants are incubated on the plates for one hour at 37° C. The plates are washed and probed with HRP labeled goat anti-mouse antibody diluted 1:10,000 in 1% BSA-PBS for one hour at 37° C. The plates are then incubated with substrate solution as described above.

Determination of Anti-NIgSAb Reactivity

Hybridomas, as above, can be simultaneously assayed for reactivity to native immunoglobulin using a suitable RIA Western blot or other assay. The anti-NIgSAb secreting hybridomas can be expanded in cell culture and serially subcloned by limiting dilution. The resulting clonal populations can be expanded and cryopreserved in freezing medium (95% FBS, 5% DMSO) and stored in liquid nitrogen. Monoclonal antibodies of the present invention will demonstrate specificity for the native immunoglobulin compared to denatured immunoglobulin.

Class Typing and Isotyping

Isotype determination of the antibodies can be accomplished using an EIA in a format similar to that used to screen the mouse immune sera for specific titers. The antigen can be coated on 96-well plates as described above and purified antibody at 2 μg/mL can be incubated on the plate for one hour at room temperature. The plate is washed and probed with HRP labeled goat anti-mouse IgG₁ or HRP labeled goat anti-mouse IgG₃ or any other class-specific or isotype-specific antibody, diluted at 1:4000 in 1% BSA-PBS for one hour at room temperature. The plate is again washed and incubated with substrate solution as described above.

Results and Discussion

Generation of Anti-NIgSAb Monoclonal Antibodies

Several fusions are performed and each fusion is seeded in fifteen plates (1440 wells/fusion) that yield several dozen antibodies specific for native immunoglobulin. Several fusions are performed utilizing splenocytes from mice that are immunized with immunoglobulin protein. A set of several native immunoglobulin-reactive monoclonal antibodies are generated. The anti-NIgSAb are further characterized to demonstrate specific binding to native immunoglobulin compared to denatured immunoglobulin.

Citations

The following references are entirely incorporated herein by reference: Ausubel et al. (Ed.), Current Protocols in Molecular Biology, (John Wiley & Sons, Inc., New York, N.Y. (1987-1991)); Sambrook et al., Molecular Cloning: A Laboratory Manual, 2^{nd} Edition, (Cold Spring Harbor, N.Y. (1989)), and Sambrook et al., Molecular Cloning—A Laboratory Manual (3rd Ed.), Vol. 1-3, Cold Spring Harbor Laboratory, Cold Spring Harbor, N.Y., (2000); Harlow and Lane, Antibodies, A Laboratory Manual, (Cold Spring Harbor, N.Y. (1989)); Colligan et al. (Eds.), Current Protocols in Immunology, (John Wiley & Sons, Inc., NY (1994-2001)); Colligan et al., Current Protocols in Protein Science, (John Wiley & Sons, NY, N.Y., (1997-2001)).

The following references provide useful details regarding procedures useful in conjunction with the present invention, and are also incorporated entirely by reference herein.

(1.) Kohler, G. and C. Milstein, “Continuous Cultures of Fused Cells Secreting Antibody of Predefined Specificity”, NATURE, (1975), 256(5517):495-7.

(2.) Harlow, E. and D. Lane, Antibodies: A Laboratory Manual, Cold Spring Harbor Press, 1988.

(3.) “Lymphocyte Hybridomas”, Current Topics in Microbiology and Immunology, Volume 81 (F. Melchers, M. Potter, and N. Warner, Editors, Springer-Verlag, 1978).

(4.) Brooks et al., “Subtractive Immunization Yields Monoclonal Antibodies that Specifically Inhibit Metastasis”, J. OF CELL BIOL., (1993) 122(6):1351-1359.

(5.) Williams, C. V., C. L. Stechmann and S. C. McLoon, “Subtractive Immunization Techniques for the Production of Monoclonal Antibodies to Rare Antigens”, BIOTECHNIQUES, (1992) 12(6):842-847.

(6.) Sleister, H. M. and A. G. Rao, “Subtractive Immunization: A Tool for the Generation of Discriminatory Antibodies to Proteins of Similar Sequence”, J. IMMUNOL. METHODS., (2002) 261(1-2):213-220.

All publications, patents or other documents cited herein are entirely incorporated herein by reference, as though each publication, patent or other document were specifically indicated to be incorporated by reference in its entirety. These documents show the state of the art at the time of the present invention and/or provide description and enablement of the present invention. Publications include any scientific or patent publications, or any other information available in any media format, including all recorded, electronic or printed formats.

1. An isolated or recombinant anti-NigSAb.

2. The anti-NIgSAb of claim 1, wherein said anti-NIgSAb specifically binds to a native immunoglobulin protein.

3. The anti-NIgSAb of claim 2, wherein said antibody is selected from a group consisting of: a monoclonal antibody, a polyclonal antibody, an antibody portion, an antibody fragment, an antibody variant, an anti-NIgSAb, an anti-NIgSAb portion, an anti-NIgSAb fragment, and an anti-NIgSAb variant.

4. The antibody of claim 3, wherein said antibody is raised in a mammal.

5. The antibody of claim 4, wherein said mammal is selected from a group consisting of: a human, a mouse, a rat, a goat, a rabbit, a sheep, a horse and a hamster.

6. The polyclonal antibody of claim 2, wherein said polyclonal antibody was raised in a mammal by subtractive immunization with a denatured immunoglobulin followed by immunization with the native immunoglobulin.

7. The polyclonal antibody of claim 2, wherein said polyclonal antibody is treated to remove an anti-denatured immunoglobulin antibody by contacting said polyclonal antibody with a denatured immunoglobulin attached to an insoluble substrate.

8. The polyclonal antibody of claim 7, wherein said insoluble substrate is selected from a group consisting of: a bead, a plate, a well, a tube, a membrane or a sheet.

9. The anti-NIgSAb of claim 1, wherein said anti-NIgSAb is produced by a recombinant animal or recombinant host cell.

10. The anti-NIgSAb of claim 9, wherein said recombinant host cell is selected from a group consisting of: a mammalian cell, an insect cell, a yeast cell, and a bacterial cell.

11. The anti-NIgSAb of claim 14, wherein said anti-NIgSAb is produced by a bacteriophage.

12. A recombinant cell line that expresses an anti-NigSAb.

13. A composition comprising at least one anti-NIgSAb and a diluent or carrier.

14. A method for producing at least one anti-NIgSAb, the method comprising: screening antibodies raised against an immunoglobulin antigen to identify antibodies that bind specifically to a native immunoglobulin.

15. The method of claim 14, wherein said anti-NIgSAb is selected from a group consisting of: a polyclonal antibody, a monoclonal antibody, an antibody portion, an antibody fragment, an antibody variant, an anti-NIgSAb fragment, an anti-NIgSAb portion and an anti-NIgSAb variant.

16. An article of manufacture for research or diagnostic use, comprising packaging material and a container comprising at least one isolated anti-NIgSAb.

17. The article of claim 16, wherein said anti-NIgSAb is detectably labeled.

18. The article of claim 17, wherein said label is selected from a group consisting of: a chemiluminescent agent, a radioisotope, an enzyme, a fluorescent agent and a chromogenic agent.

19. The article of claim 18, wherein said label comprises HRP.

20. The article of claim 17, wherein said antibody is raised in a mammal selected from the group consisting of: a mouse, a rat, a rabbit, a goat, a hamster and a horse.

21. A method for detecting native immunoglobulin, the method comprising: contacting a sample with one or more anti-NIgSAb, and measuring binding of said anti-NIgSAb to said sample.

22. The method of claim 21, wherein said anti-NIgSAb is detectably labeled.

23. The method of claim 22,wherein said anti-NIgSAb is an enzyme that catalyzes formation of a detectable reaction product.

24. The method of claim 21, wherein said sample is immobilized on a substrate.

25. The method of claim 24, wherein said substrate is selected from a group consisting of: a bead, a plate, a sheet, a strip, a well, a membrane and a tube.

26. An isolated or recombinant NIgSBR.

27. The NIgSBR of claim 26, wherein the NIgSBR is detectably labeled.

28. The NIgSBR of claim 27, wherein the label is selected from a group consisting of: a chemiluminescent agent, a radioisotope, an enzyme, a fluorescent agent, and a chromogenic agent.

29. A composition comprising at least one NIgSBR and a suitable diluent or carrier.

30. An article of manufacture for research or diagnostic use, comprising packaging materials and a container comprising at least one NIgSBR.

31. A method for producing at least one NIgSBR comprising screening one or more compound to identify a compound that specifically binds to a native immunoglobulin.

32. The method of claim 31, wherein said NIgSBR is identified from a library selected from a group consisting of: a protein scaffold library, a peptide display library, a directed evolution library, and a protein array-based library.

33. The method of claim 31, wherein said NIgSBR is selected from a group consisting of: a polyclonal antibody, a monoclonal antibody, an antibody portion, an antibody fragment, an antibody variant, an engineered protein, a polymer scaffold, an engineered compound, a polypeptide, and a polymer made in a mammalian system, a polymer made in a non-mammalian system, and a polymer made in an E. coli by phage display.

34. A method for detecting a native immunoglobulin, the method comprising: contacting a sample with one or more NIgSBR, and measuring binding of said NIgSBR to said sample.

35. The method of claim 34, wherein said NIgSBR is detectably labeled.

36. The method of claim 34, wherein said sample is immobilized on a substrate.

37. The method of claim 36, wherein said substrate is selected from a group consisting of: a bead, a plate, a sheet, a strip, a well and a tube.
